(19) United States
(12) Patent Application Publication
(10) Pub. No.: US 2021/0130496 A1
(43) Pub. Date: May 6, 2021

(54) MULTISPECIFIC BINDING PROTEINS TARGETING CEA

(71) Applicant: Dragonfly Therapeutics, Inc., Waltham, MA (US)

(72) Inventors: Gregory P. Chang, Medford, MA (US); Ann F. Cheung, Lincoln, MA (US); Eva Gutierrez, Waltham, MA (US); William Haney, Wayland, MA (US); Bradley M. Lunde, Lebanon, NH (US); Bianka Prinz, Lebanon, NH (US)

(21) Appl. No.: 16/488,395

(22) PCT Filed: Feb. 27, 2018
(86) PCT No.: PCT/US2018/019999

§ 371 Date: Aug. 23, 2019

Related U.S. Application Data
(60) Provisional application No. 62/473,652, filed on Mar. 20, 2017.
(60) Provisional application No. 62/473,659, filed on Mar. 20, 2017.
(60) Provisional application No. 62/467,557, filed on Mar. 6, 2017.
(60) Provisional application No. 62/464,341, filed on Feb. 27, 2017.
(60) Provisional application No. 62/464,347, filed on Feb. 27, 2017.
(60) Provisional application No. 62/464,344, filed on Feb. 27, 2017.

(51) Int. Cl. C07K 16/46; C07K 16/28

(52) U.S. Cl. CPC C07K 16/46; C07K 16/283; C07K 2317/569; C07K 2317/524; C07K 2317/53; C07K 2317/73

(57) ABSTRACT

Multi-specific binding proteins that bind the NKG2D receptor, CD 16, and a tumor-associated antigen selected from carbonic anhydrase 9 (CAIX), anoctamin-1 (ANO1), mesothelin, TROP2, CEA and claudin-18.2 are described, as well as pharmaceutical compositions and therapeutic methods useful for the treatment of cancer.

### CROSS-REFERENCE TO RELATED APPLICATIONS

This application claims the benefit of and priority to U.S. application No. 62/464,341 filed Feb. 27, 2017, U.S. application No. 62/464,344 filed Feb. 27, 2017, U.S. application No. 62/464,347 filed Feb. 27, 2017, U.S. application No. 62/467,557 filed Mar. 6, 2017, U.S. application No. 62/473,652 filed Mar. 20, 2017, and U.S. application No. 62/473,659 filed Mar. 20, 2017, the entire contents of each of which are hereby incorporated by reference in their entireties for all purposes.

### SEQUENCE LISTING

The instant application contains a Sequence Listing which has been submitted electronically in ASCII format and is hereby incorporated by reference in its entirety. Said ASCII copy, created on Feb. 27, 2018, is named DFY-012WO_ST25.txt and is 212,480 bytes in size.

### FIELD OF THE INVENTION

The invention relates to multi-specific binding proteins that bind to the NKG2D receptor, CD16, and a tumor-associated antigen selected from carbonic anhydrase 9 (CAIX), anoctamin-1 (ANO1), mesothelin, trophoblast cell-surface antigen (TROP2), carcinoembryonic antigen (CEA) and Claudin-18.2.

### BACKGROUND

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Small cell lung cancer remains among the most deadly of malignancies. Current treatment options for these cancers are not effective for all patients and/or can have substantial adverse side effects. Other types of cancer also remain challenging to treat using existing therapeutic options.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Antibodies that bind to certain tumor-associated antigens and to certain immune cells have been described in the literature. See, e.g., WO 2016/134371 and WO 2015/095412.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e., via cytolytic granules that contain perforin and granzymes as well as via death receptor pathways. Activated NK cells also secrete inflammatory cytokines such as IFN-γ and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD16 receptors on their surface. The overall sensitivity of NK cells to activation depends on the sum of stimulatory and inhibitory signals.

Carbonic anhydrase 9 (CAIX) is a zinc metalloenzyme and belongs to a family of enzymes that are involved in reversible hydration of carbon dioxide to form bicarbonate and hydrogen ions. CAIX has limited expression in normal tissues, but is overexpressed in a wide variety of solid tumors and plays an important role in tumor development. Induced by hypoxic tumor microenvironments, CAIX functions to combat the deleterious effects of a high rate of glycolytic metabolism in tumor cells by maintaining an intracellular pH favorable for tumor cell growth and survival, while at the same time generating an acidic extracellular space facilitating tumor cell invasiveness. So far, overexpression of CAIX has been detected in renal cell carcinoma, breast cancer, glioblastoma, head and neck cancer, gastric cancers, bladder cancer, ovarian cancer, as well as in common epithelial cancers such as carcinomas of the esophagus, lung, colon, kidney, cervix and non-small cell lung carcinoma.

Mesothelin is a glycosylphosphatidylinositol (GPI) anchored cell surface protein, and is expressed in mesothelial cells of the pleura, peritoneum, and pericardium. In addition to its normal expression, mesothelin is overexpressed in a variety of cancers, including mesothelioma, ovarian cancer, pancreatic cancer, non-small cell lung cancer, breast cancer, cholangiocarcinoma, gastric cancer, uterine serous carcinoma, thymic carcinoma, and acute myeloid leukemia. It is suggested that mesothelin may play an important role in tumor progression including cell adherence, cell survival/proliferation and chemoresistance.

Anoctamin-1 (ANO1) is a voltage-sensitive calcium-activated chloride channel. It is also known by other names, such as TMEM16, TAOS2, ORAOV2, and DOG-1. Calcium-activated chloride channels function in many physiological processes, including transepithelial secretion, cardiac and neuronal excitation, sensory transduction, smooth muscle contraction, and fertilization. ANO1 is potentially involved in epithelial fluid secretion, olfactory and phototransduction, neuronal and cardiac excitability, and regulation of vascular tone including gut motility. ANO1 is also highly expressed in some cancers, where it is thought to augment cell proliferation, cell migration and metastasis, for example, in esophageal squamous cell cancer (ESCC), gastrointestinal stromal tumor (GIST), head and neck squamous cell carcinoma (HNSCC), pancreatic cancer, breast cancer, prostate cancer, and sarcoma. ˜85% of HNSCC tumors are found to be ANO1 positive, and HNSCC patients with high-level tumor expression of ANO1 have decreased overall survival; overexpression of ANO1 is also found to be closely correlated with malignancy of prostate cancer and poor prognosis of breast cancer.

TROP2 is a transmembrane glycoprotein and a Ca²⁺-signal transducer. In addition to its expression in certain normal tissues, TROP2 is overexpressed in a variety of cancers including breast, lung, gastric, colorectal, pancreatic, prostatic, cervical, head-and-neck cancer, nasopharyngeal carcinoma, and ovarian carcinoma. It signals cancer cells for proliferation, invasion, and survival.

Carcinoembryonic antigen (CEA), also known as CD66e or CEACAM5 is a member of the immunoglobulin superfamily. It is a large cell surface glycoprotein, and mainly serves as a cell adhesion molecule mediating intercellular contact. Besides its functions in cell adhesion and migration, CEA is found to be over-expressed in a high percentage of human cancers, including 90% of gastrointestinal, colorectal and pancreatic cancers, 70% of non-small cell lung cancer cells and 50% of breast cancers. Overexpression of CEA has been shown to correlate with tumorigenicity and enhanced tumor invasiveness.

Claudin-18.2 is one of the two isoforms Claudin-18, a major component of tight junctions which regulate permeability, barrier function and polarity of epithelial layers. Claudin-18.2 is expressed only in short-lived differentiated cells of the gastric mucosa in healthy tissues. However, it is overexpressed in up to 80% of gastrointestinal adenocarcinomas and 60% pancreatic tumors. In addition, its expression has been observed in distinct subsets of individuals with esophageal cancer, non-small cell lung carcinoma, ovarian cancer, colon cancer, and several forms of biliary ductal carcinoma.

### SUMMARY

The invention provides multi-specific binding proteins that bind to CAIX, ANO1, mesothelin, TROP2, CEA or Claudin-18.2 on a cancer cell and to the NKG2D receptor and CD16 receptor on natural killer cells. Such proteins can engage more than one kind of NK activating receptor, and may block the binding of natural ligands to NKG2D. In certain embodiments, the proteins can agonize NK cells in humans, and in other species such as rodents and cynomolgus monkeys. Various aspects and embodiments of the invention are described in further detail below.

Accordingly, one aspect of the invention provides a protein that incorporates a first antigen-binding site that binds NKG2D; a second antigen-binding site that binds CAIX, ANO1, mesothelin, TROP2, CEA or Claudin-18.2; and an antibody Fc domain, a portion thereof sufficient to bind CD16, or a third antigen-binding site that binds CD16. The antigen-binding sites may each incorporate an antibody heavy chain variable domain and an antibody light chain variable domain (e.g. arranged as in an antibody, or fused together to from an scFv), or one or more of the antigen-binding sites may be a single domain antibody, such as a V_{H}H antibody like a camelid antibody or a V_{NAR} antibody like those found in cartilaginous fish.

In one embodiment, the first antigen-binding site, which binds to NKG2D, incorporates a heavy chain variable domain related to SEQ ID NO:1, such as by having an amino acid sequence at least 90% (e.g., 90%, 91%, 92%, 93%, 94%, 95%, 96%, 97%, 98%, 99%, or 100%) identical to SEQ ID NO:1, and/or incorporating amino acid sequences identical to the CDR1 (SEQ ID NO:51), CDR2 (SEQ ID NO:52), and CDR3 (SEQ ID NO:53) sequences of SEQ ID NO:1. The heavy chain variable domain related to SEQ ID NO:1 can be coupled a variety of light chain variable domains to form a NKG2D binding site. For example, the first antigen-binding site that incorporates a heavy chain variable domain related to SEQ ID NO:1 can further incorporate a light chain variable domain selected from any one of the sequences related to SEQ ID NOs:2, 4, 6, 8, 10, 12, 14, 16, 18, 20, 22, 24, 26, 28, 30, 32, 34, 36, and 40. For example, the first antigen-binding site incorporates a heavy chain variable domain with amino acid sequences at least 90% (e.g., 90%, 91%, 92%, 93%, 94%, 95%, 96%, 97%, 98%, 99%, or 100%) identical to SEQ ID NO:1 and a light chain variable domain with amino acid sequences at least 90% (e.g., 90%, 91%, 92%, 93%, 94%, 95%, 96%, 97%, 98%, 99%, or 100%) identical to any one of the sequences selected from SEQ ID NOs:2, 4, 6, 8, 10, 12, 14, 16, 18, 20, 22, 24, 26, 28, 30, 32, 34, 36, and 40. Alternatively, the first antigen-binding site can incorporate a heavy chain variable domain related to SEQ ID NO:41 and a light chain variable domain related to SEQ ID NO:42. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:41, and/or incorporate amino acid sequences identical to the CDR1 (SEQ ID NO:54), CDR2 (SEQ ID NO:55), and CDR3 (SEQ ID NO:56) sequences of SEQ ID NO:41. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:42, and/or incorporate amino acid sequences identical to the CDR1 (SEQ ID NO:57), CDR2 (SEQ ID NO:58), and CDR3 (SEQ ID NO:59) sequences of SEQ ID NO:42. In other embodiments, the first antigen-binding site can incorporate a heavy chain variable domain related to SEQ ID NO:43 and a light chain variable domain related to SEQ ID NO:44. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:43, and/or incorporate amino acid sequences identical to the CDR1 (SEQ ID NO:60), CDR2 (SEQ ID NO:61), and CDR3 (SEQ ID NO:62) sequences of SEQ ID NO:43. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:44, and/or incorporate amino acid sequences identical to the CDR1 (SEQ ID NO:63), CDR2 (SEQ ID NO:64), and CDR3 (SEQ ID NO:65) sequences of SEQ ID NO:44. In certain embodiments, the first antigen-binding site can incorporate a heavy chain variable domain related to SEQ ID NO:45 and a light chain variable domain related to SEQ ID NO:46. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:45, and/or incorporate amino acid sequences identical to the CDR1 (SEQ ID NO:66), CDR2 (SEQ ID NO:67), and CDR3 (SEQ ID NO:68) sequences of SEQ ID NO:45. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:46, and/or incorporate amino acid sequences identical to the CDR1 (SEQ ID NO:69), CDR2 (SEQ ID NO:70), and CDR3 (SEQ ID NO:71) sequences of SEQ ID NO:46.

Alternatively, the first antigen-binding site can incorporate a heavy chain variable domain related to SEQ ID NO:47 and a light chain variable domain related to SEQ ID NO:48, such as by having amino acid sequences at least 90% (e.g., 90%, 91%, 92%, 93%, 94%, 95%, 96%, 97%, 98%, 99%, or 100%) identical to SEQ ID NO:47 and SEQ ID NO:48 respectively. In another embodiment, the first antigen-binding site can incorporate a heavy chain variable domain related to SEQ ID NO:49 and a light chain variable domain related to SEQ ID NO:50, such as by having amino acid sequences at least 90% (e.g., 90%, 91%, 92%, 93%, 94%, 95%, 96%, 97%, 98%, 99%, or 100%) identical to SEQ ID NO:49 and SEQ ID NO:50 respectively.

In some embodiments, the first antigen-binding site can incorporate a heavy chain variable domain related to SEQ ID NO:132 and a light chain variable domain related to SEQ ID NO:133. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:132, and/or incorporate amino acid sequences identical to the CDR1 (SEQ ID NO:134), CDR2 (SEQ ID NO:135), and CDR3 (SEQ ID NO:136) sequences of SEQ ID NO:132. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:133, and/or incorporate amino acid sequences identical to the CDR1 (SEQ ID NO:137), CDR2 (SEQ ID NO:138), and CDR3 (SEQ ID NO:139) sequences of SEQ ID NO:133.

In some embodiments, the first antigen-binding site can incorporate a heavy chain variable domain related to SEQ ID NO:140 and a light chain variable domain related to SEQ ID NO:141. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:140, and/or incorporate amino acid sequences identical to the CDR1 (SEQ ID NO:142), CDR2 (SEQ ID NO:143), and CDR3 (SEQ ID NO:144) sequences of SEQ ID NO:140. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:141, and/or incorporate amino acid sequences identical to the CDR1 (SEQ ID NO:145), CDR2 (SEQ ID NO:146), and CDR3 (SEQ ID NO:147) sequences of SEQ ID NO:141.

In some embodiments, the first antigen-binding site can incorporate a heavy chain variable domain related to SEQ ID NO:148 and a light chain variable domain related to SEQ ID NO:149. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:148, and/or incorporate amino acid sequences identical to the CDR1 (SEQ ID NO:150), CDR2 (SEQ ID NO:151), and CDR3 (SEQ ID NO:152) sequences of SEQ ID NO:148. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:149, and/or incorporate amino acid sequences identical to the CDR1 (SEQ ID NO:153), CDR2 (SEQ ID NO:154), and CDR3 (SEQ ID NO:155) sequences of SEQ ID NO:149.

In some embodiments, the first antigen-binding site can incorporate a heavy chain variable domain related to SEQ ID NO:156 and a light chain variable domain related to SEQ ID NO:157. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:156, and/or incorporate amino acid sequences identical to the CDR1 (SEQ ID NO:158), CDR2 (SEQ ID NO:159), and CDR3 (SEQ ID NO:160) sequences of SEQ ID NO:156. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:157, and/or incorporate amino acid sequences identical to the CDR1 (SEQ ID NO:161), CDR2 (SEQ ID NO:162), and CDR3 (SEQ ID NO:163) sequences of SEQ ID NO:157.

In some embodiments, the first antigen-binding site can incorporate a heavy chain variable domain related to SEQ ID NO:164 and a light chain variable domain related to SEQ ID NO:165. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:164, and/or incorporate amino acid sequences identical to the CDR1 (SEQ ID NO:166), CDR2 (SEQ ID NO:167), and CDR3 (SEQ ID NO:168) sequences of SEQ ID NO:164. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:165, and/or incorporate amino acid sequences identical to the CDR1 (SEQ ID NO:169), CDR2 (SEQ ID NO:170), and CDR3 (SEQ ID NO:171) sequences of SEQ ID NO:165.

The second antigen binding site can bind to CAIX, ANO1, mesothelin, TROP2, CEA or Claudin-18.2. In some embodiments, the second antigen binding site binds to CAIX and incorporates a heavy chain variable domain and light chain variable domain that can be coupled to form a CAIX binding site. For example, the second antigen binding site can incorporate a heavy chain variable domain related to SEQ ID NO:72 and a light chain variable domain related to SEQ ID NO:73. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:72, and/or incorporate amino acid sequences identical to the CDR1 (SEQ ID NO:74), CDR2 (SEQ ID NO:75), and CDR3 (SEQ ID NO:76) sequences of SEQ ID NO:72. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:73, and/or incorporate amino acid sequences identical to the CDR1 (SEQ ID NO:77), CDR2 (SEQ ID NO:78), and CDR3 (SEQ ID NO:79) sequences of SEQ ID NO:73.

Alternatively, the second antigen binding site can incorporate a heavy chain variable domain related to SEQ ID NO:80 and a light chain variable domain related to SEQ ID NO:81. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:80, and/or incorporate amino acid sequences identical to the CDR1 (SEQ ID NO:82), CDR2 (SEQ ID NO:83), and CDR3 (SEQ ID NO:84) sequences of SEQ ID NO:80. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:81, and/or incorporate amino acid sequences identical to the CDR1 (SEQ ID NO:85), CDR2 (SEQ ID NO:86), and CDR3 (SEQ ID NO:87) sequences of SEQ ID NO:81.

Alternatively, the second antigen binding site can incorporate a heavy chain variable domain related to SEQ ID NO:88 and a light chain variable domain related to SEQ ID NO:89. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:88, and/or incorporate amino acid sequences identical to the CDR1 (SEQ ID NO:90), CDR2 (SEQ ID NO:91), and CDR3 (SEQ ID NO:92) sequences of SEQ ID NO:88. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:89, and/or incorporate amino acid sequences identical to the CDR1 (SEQ ID NO:93), CDR2 (SEQ ID NO:94), and CDR3 (SEQ ID NO:95) sequences of SEQ ID NO:89.

In some embodiments, the second antigen binding site binds to ANO1 and incorporates a heavy chain variable domain and light chain variable domain that can be coupled to form an ANO1 binding site. For example, the second antigen binding site can incorporate a variety a heavy chain variable domain related to SEQ ID NO:97 and a light chain variable domain related to SEQ ID NO:98. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:97, and/or incorporate amino acid sequences identical to the CDR1 (SEQ ID NO:99), CDR2 (SEQ ID NO:100), and CDR3 (SEQ ID NO:101) sequences of SEQ ID NO:97. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:102), CDR2 (SEQ ID NO:103), and CDR3 (SEQ ID NO:104) sequences of SEQ ID NO:98.

In some embodiments, the second antigen binding site binds to mesothelin and incorporates a heavy chain variable domain and light chain variable domain that can be coupled to form a mesothelin binding site. For example, the second antigen binding site can incorporate a variety a heavy chain variable domain related to SEQ ID NO:106 and a light chain variable domain related to SEQ ID NO:107. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:106, and/or incorporate amino acid sequences identical to the CDR1 (SEQ ID NO:108), CDR2 (SEQ ID NO:109), and CDR3 (SEQ ID NO:110) sequences of SEQ ID NO:106. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:107, and/or incorporate amino acid sequences identical to the CDR1 (SEQ ID NO:111), CDR2 (SEQ ID NO:112), and CDR3 (SEQ ID NO:113) sequences of SEQ ID NO:107. Alternatively, the second antigen binding site can incorporate a variety a heavy chain variable domain related to SEQ ID NO:114 and a light chain variable domain related to SEQ ID NO:115. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:114, and/or incorporate amino acid sequences identical to the CDR1 (SEQ ID NO:116), CDR2 (SEQ ID NO:117), and CDR3 (SEQ ID NO:118) sequences of SEQ ID NO:115. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:115, and/or incorporate amino acid sequences identical to the CDR1 (SEQ ID NO:119), CDR2 (SEQ ID NO:120), and CDR3 (SEQ ID NO:121) sequences of SEQ ID NO:115. Alternatively, the second antigen binding site can incorporate a variety a heavy chain variable domain related to SEQ ID NO:122 and a light chain variable domain related to SEQ ID NO:123. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:122, and/or incorporate amino acid sequences identical to the CDR1 (SEQ ID NO:124), CDR2 (SEQ ID NO:125), and CDR3 (SEQ ID NO:126) sequences of SEQ ID NO:122. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:123, and/or incorporate amino acid sequences identical to the CDR1 (SEQ ID NO:127), CDR2 (SEQ ID NO:128), and CDR3 (SEQ ID NO:129) sequences of SEQ ID NO:123.

In some embodiments, the second antigen binding site binds to TROP2 and incorporates a heavy chain variable domain and light chain variable domain that can be coupled to form a TROP2 binding site. For example, the second antigen binding site can incorporate a heavy chain variable domain related to SEQ ID NO:172 and a light chain variable domain related to SEQ ID NO:173. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:172, and/or incorporate amino acid sequences identical to the CDR1 (SEQ ID NO:174), CDR2 (SEQ ID NO:175), and CDR3 (SEQ ID NO:176) sequences of SEQ ID NO:172. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:173, and/or incorporate amino acid sequences identical to the CDR1 (SEQ ID NO:177), CDR2 (SEQ ID NO:178), and CDR3 (SEQ ID NO:179) sequences of SEQ ID NO:173. Alternatively, the second antigen binding site can incorporate a heavy chain variable domain related to SEQ ID NO:180 and a light chain variable domain related to SEQ ID NO:181. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:180, and/or incorporate amino acid sequences identical to the CDR1 (SEQ ID NO:182), CDR2 (SEQ ID NO:183), and CDR3 (SEQ ID NO:184) sequences of SEQ ID NO:180. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:181, and/or incorporate amino acid sequences identical to the CDR1 (SEQ ID NO:185), CDR2 (SEQ ID NO:186), and CDR3 (SEQ ID NO:187) sequences of SEQ ID NO:181. Alternatively, the second antigen binding site can incorporate a heavy chain variable domain related to SEQ ID NO:188 and a light chain variable domain related to SEQ ID NO:189. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:189, and the light chain variable domain of the second antigen binding site can be at least 90% (e.g., 90%, 91%, 92%, 93%, 94%, 95%, 96%, 97%, 98%, 99%, or 100%) identical to SEQ ID NO:189. Alternatively, the second antigen binding site can incorporate a heavy chain variable domain related to SEQ ID NO:190 and a light chain variable domain related to SEQ ID NO:191. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:190, and the light chain variable domain of the second antigen binding site can be at least 90% (e.g., 90%, 91%, 92%, 93%, 94%, 95%, 96%, 97%, 98%, 99%, or 100%) identical to SEQ ID NO:191. Alternatively, the second antigen binding site can incorporate a heavy chain variable domain related to SEQ ID NO:192 and a light chain variable domain related to SEQ ID NO:193. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:192, and the light chain variable domain of the second antigen binding site can be at least 90% (e.g., 90%, 91%, 92%, 93%, 94%, 95%, 96%, 97%, 98%, 99%, or 100%) identical to SEQ ID NO:193.

In some embodiments, the second antigen binding site binds to CEA and incorporates a heavy chain variable domain and light chain variable domain that can be coupled to form a CEA binding site. For example, the second antigen binding site can incorporate a heavy chain variable domain related to SEQ ID NO:195 and a light chain variable domain related to SEQ ID NO:196. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:195, and/or incorporate amino acid sequences identical to the CDR1 (SEQ ID NO:197), CDR2 (SEQ ID NO:198), and CDR3 (SEQ ID NO:199) sequences of SEQ ID NO:195. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:196, and/or incorporate amino acid sequences identical to the CDR1 (SEQ ID NO:200), CDR2 (SEQ ID NO:201), and CDR3 (SEQ ID NO:202) sequences of SEQ ID NO:196. Alternatively, the second antigen binding site can incorporate a heavy chain variable domain related to SEQ ID NO:203 and a light chain variable domain related to SEQ ID NO:204. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:203, and/or incorporate amino acid sequences identical to the CDR1 (SEQ ID NO:205), CDR2 (SEQ ID NO:206), and CDR3 (SEQ ID NO:207) sequences of SEQ ID NO:203. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:204, and/or incorporate amino acid sequences identical to the CDR1 (SEQ ID NO:208), CDR2 (SEQ ID NO:209), and CDR3 (SEQ ID NO:210) sequences of SEQ ID NO:204. Alternatively, the second antigen binding site can incorporate a heavy chain variable domain related to SEQ ID NO:211 and a light chain variable domain related to SEQ ID NO:212. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:211, and/or incorporate amino acid sequences identical to the CDR1 (SEQ ID NO:213), CDR2 (SEQ ID NO:214), and CDR3 (SEQ ID NO:215) sequences of SEQ ID NO:212. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:196, and/or incorporate amino acid sequences identical to the CDR1 (SEQ ID NO:216), CDR2 (SEQ ID NO:217), and CDR3 (SEQ ID NO:218) sequences of SEQ ID NO:212.

In some embodiments, the second antigen binding site binds to Claudin-18.2 and incorporates a heavy chain variable domain and light chain variable domain that can be coupled to form a Claudin-18.2 binding site. For example, the second antigen binding site can incorporate a heavy chain variable domain related to SEQ ID NO:220 and a light chain variable domain related to SEQ ID NO:221. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:220, and/or incorporate amino acid sequences identical to the CDR1 (SEQ ID NO:222), CDR2 (SEQ ID NO:223), and CDR3 (SEQ ID NO:224) sequences of SEQ ID NO:220. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:221, and/or incorporate amino acid sequences identical to the CDR1 (SEQ ID NO:225), CDR2 (SEQ ID NO:226), and CDR3 (SEQ ID NO:227) sequences of SEQ ID NO:221. For example, the second antigen binding site can incorporate a heavy chain variable domain related to SEQ ID NO:228 and a light chain variable domain related to SEQ ID NO:229. For example, the heavy chain variable domain of the second antigen binding site can be at least 90% (e.g., 90%, 91%, 92%, 93%, 94%, 95%, 96%, 97%, 98%, 99%, or 100%) identical to SEQ ID NO:228, and/or incorporate amino acid sequences identical to the CDR1 (SEQ ID NO:230), CDR2 (SEQ ID NO:231), and CDR3 (SEQ ID NO:232) sequences of SEQ ID NO:228. Similarly, the light chain variable domain of the second antigen binding site can be at least 90% (e.g., 90%, 91%, 92%, 93%, 94%, 95%, 96%, 97%, 98%, 99%, or 100%) identical to SEQ ID NO:229, and/or incorporate amino acid sequences identical to the CDR1 (SEQ ID NO:233), CDR2 (SEQ ID NO:234), and CDR3 (SEQ ID NO:235) sequences of SEQ ID NO:229.

In some embodiments, the second antigen binding site incorporates a light chain variable domain having an amino acid sequence identical to the amino acid sequence of the light chain variable domain present in the first antigen binding site.

In some embodiments, the protein incorporates a portion of an antibody Fc domain sufficient to bind CD16, wherein the antibody Fc domain comprises hinge and CH2 domains, and/or amino acid sequences at least 90% identical to amino acid sequence 234-332 of a human IgG antibody.

Formulations containing one of these proteins; cells containing one or more nucleic acids expressing these proteins, and methods of enhancing tumor cell death using these proteins are also provided.

Another aspect of the invention provides a method of treating cancer in a patient. The method comprises administering to a patient in need thereof a therapeutically effective amount of the multi-specific binding proteins described herein. Cancers to be treated using CAIX-targeting multi-specific binding proteins include, for example, renal cell carcinoma, breast cancer, glioblastoma, head and neck cancer, gastric cancers, bladder cancer, ovarian cancer, tumors of the esophagus, lung, colon, kidney, cervix and non-small cell lung carcinoma. Cancers to be treated using mesothelin-targeting multi-specific binding proteins include, for example, mesothelioma, ovarian cancer, pancreatic cancer, non-small cell lung cancer, breast cancer, cholangiocarcinoma, gastric cancer, uterine serous carcinoma, thymic carcinoma, and acute myeloid leukemia. Cancers to be treated using ANO1-targeting multi-specific binding proteins include, for example, esophageal squamous cell cancer (ESCC), gastrointestinal stromal tumor (GIST), head and neck squamous cell carcinoma (HNSCC), pancreatic cancer, breast cancer, prostate cancer, and sarcoma. Cancers to be treated using TROP2-targeting multi-specific binding proteins include, for example, breast, lung, gastric, colorectal, pancreatic, prostatic, cervical, head-and-neck cancer, nasopharyngeal carcinoma, and ovarian carcinoma. Cancers to be treated using CEA-targeting multi-specific binding proteins include, for example, gastrointestinal cancer, colorectal cancer, pancreatic cancer, non-small cell lung cancer and breast cancer. Cancers to be treated using claudin-18.2-targeting multi-specific binding proteins include, for example, esophageal cancer, non-small cell lung carcinoma, ovarian cancer, colon cancer, and several forms of biliary ductal carcinoma.

### BRIEF DESCRIPTION OF THE DRAWINGS

FIG. 1 is a representation of a heterodimeric, multi-specific antibody. Each arm can represent either the NKG2D-binding domain or one of the tumor associated antigen-binding domain selected from CAIX, ANO1, mesothelin, TROP2, CEA and Claudin-18.2 binding domains. In some embodiments, the NKG2D- and the tumor associated antigen-binding domains can share a common light chain.

FIG. 2 is a representation of a heterodimeric, multi-specific antibody. Either the NKG2D- or the tumor associated antigen-binding domain selected from CAIX, ANO1, mesothelin, TROP2, CEA and Claudin-18.2 binding domains can take the scFv format (right arm).

FIG. 3 are line graphs demonstrating the binding affinity of NKG2D-binding domains (listed as clones) to human recombinant NKG2D in an ELISA assay.

FIG. 4 are line graphs demonstrating the binding affinity of NKG2D-binding domains (listed as clones) to cynomolgus recombinant NKG2D in an ELISA assay.

FIG. 5 are line graphs demonstrating the binding affinity of NKG2D-binding domains (listed as clones) to mouse recombinant NKG2D in an ELISA assay.

FIG. 6 are bar graphs demonstrating the binding of NKG2D-binding domains (listed as clones) to EL4 cells expressing human NKG2D by flow cytometry showing mean fluorescence intensity (MFI) fold over background.

FIG. 7 are bar graphs demonstrating the binding of NKG2D-binding domains (listed as clones) to EL4 cells expressing mouse NKG2D by flow cytometry showing mean fluorescence intensity (MFI) fold over background.

FIG. 8 are line graphs demonstrating specific binding affinity of NKG2D-binding domains (listed as clones) to recombinant human NKG2D-Fc by competing with natural ligand ULBP-6.

FIG. 9 are line graphs demonstrating specific binding affinity of NKG2D-binding domains (listed as clones) to recombinant human NKG2D-Fc by competing with natural ligand MICA.

FIG. 10 are line graphs demonstrating specific binding affinity of NKG2D-binding domains (listed as clones) to recombinant mouse NKG2D-Fc by competing with natural ligand Rae-1 delta.

FIG. 11 are bar graphs showing activation of human NKG2D by NKG2D-binding domains (listed as clones) by quantifying the percentage of TNF-alpha positive cells, which express human NKG2D-CD3 zeta fusion proteins.

FIG. 12 are bar graphs showing activation of mouse NKG2D by NKG2D-binding domains (listed as clones) by quantifying the percentage of TNF-alpha positive cells, which express mouse NKG2D-CD3 zeta fusion proteins.

FIG. 13 are bar graphs showing activation of human NK cells by NKG2D-binding domains (listed as clones).

FIG. 14 are bar graphs showing activation of human NK cells by NKG2D-binding domains (listed as clones).

FIG. 15 are bar graphs showing activation of mouse NK cells by NKG2D-binding domains (listed as clones).

FIG. 16 are bar graphs showing activation of mouse NK cells by NKG2D-binding domains (listed as clones).

FIG. 17 are bar graphs showing the cytotoxic effect of NKG2D-binding domains (listed as clones) on tumor cells.

FIG. 18 are bar graphs showing the melting temperature of NKG2D-binding domains (listed as clones) measured by differential scanning fluorimetry.

FIGS. 19A-19C are bar graphs of synergistic activation of NK cells using CD16 and NKG2D binding. FIG. 19A demonstrates levels of CD107a; FIG. 19B demonstrates levels of IFNγ; FIG. 19C demonstrates levels of CD107a and IFNγ. Graphs indicate the mean (n=2)±SD. Data are representative of five independent experiments using five different healthy donors.

FIG. 20 is a representation of a TriNKET in the Triomab form, which is a trifunctional, bispecific antibody that maintains an IgG-like shape. This chimera consists of two half antibodies, each with one light and one heavy chain, that originate from two parental antibodies. Triomab form may be a heterodimeric construct containing ½ of rat antibody and ½ of mouse antibody.

FIG. 21 is a representation of a TriNKET in the KiH Common Light Chain (LC) form, which involves the knobs-into-holes (KIHs) technology. KiH is a heterodimer containing 2 Fabs binding to target 1 and 2, and an Fc stabilized by heterodimerization mutations. TriNKET in the KiH format may be an heterodimeric construct with 2 Fabs binding to target 1 and target 2, containing two different heavy chains and a common light chain that pairs with both heavy chains.

FIG. 22 is a representation of a TriNKET in the dual-variable domain immunoglobulin (DVD-Ig™) form, which combines the target-binding domains of two monoclonal antibodies via flexible naturally occurring linkers, and yields a tetravalent IgG-like molecule. DVD-Ig™ is a homodimeric construct where variable domain targeting antigen 2 is fused to the N-terminus of a variable domain of Fab targeting antigen 1 Construct contains normal Fc.

FIG. 23 is a representation of a TriNKET in the Orthogonal Fab interface (Ortho-Fab) form, which is a heterodimeric construct that contains 2 Fabs binding to target 1 and target 2 fused to Fc. LC-HC pairing is ensured by orthogonal interface. Heterodimerization is ensured by mutations in the Fc.

FIG. 24 is a representation of a TriNKET in the 2-in-1 Ig format.

FIG. 25 is a representation of a TriNKET in the ES form, which is a heterodimeric construct containing two different Fabs binding to target 1 and target 2 fused to the Fc. Heterodimerization is ensured by electrostatic steering mutations in the Fc.

FIG. 26 is a representation of a TriNKET in the Fab Arm Exchange form: antibodies that exchange Fab arms by swapping a heavy chain and attached light chain (half-molecule) with a heavy-light chain pair from another molecule, resulting in bispecific antibodies. Fab Arm Exchange form (cFae) is a heterodimer containing 2 Fabs binding to target 1 and 2, and an Fc stabilized by heterodimerization mutations.

FIG. 27 is a representation of a TriNKET in the SEED Body form, which is an heterodimer containing 2 Fabs binding to target 1 and 2, and an Fc stabilized by heterodimerization mutations.

FIG. 28 is a representation of a TriNKET in the LuZ-Y form, in which leucine zipper is used to induce heterodimerization of two different HCs. LuZ-Y form is a heterodimer containing two different scFabs binding to target 1 and 2, fused to Fc. Heterodimerization is ensured through leucine zipper motifs fused to C-terminus of Fc.

FIG. 29 is a representation of a TriNKET in the Cov-X-Body form.

FIGS. 30A-30B are representations of TriNKETs in the κλ-Body forms, which are an heterodimeric constructs with two different Fabs fused to Fc stabilized by heterodimerization mutations: Fab1 targeting antigen 1 contains kappa LC, while second Fab targeting antigen 2 contains lambda LC. FIG. 30A is an exemplary representation of one form of a κλ-Body; FIG. 30B is an exemplary representation of another κλ-Body.

FIG. 31 is an Oasc-Fab heterodimeric construct that includes Fab binding to target 1 and scFab binding to target 2 fused to Fc. Heterodimerization is ensured by mutations in the Fc.

FIG. 32 is a DuetMab, which is a heterodimeric construct containing two different Fabs binding to antigens 1 and 2, and Fc stabilized by heterodimerization mutations. Fab1 and 2 contain differential S—S bridges that ensure correct light chain (LC) and heavy chain (HC) pairing.

FIG. 33 is a CrossmAb, which is a heterodimeric construct with two different Fabs binding to targets 1 and 2 fused to Fc stabilized by heterodimerization. CL and CH1 domains and VH and VL domains are switched, e.g., CH1 is fused in-line with VL, while CL is fused in-line with VH.

FIG. 34 is a Fit-Ig, which is a homodimeric construct where Fab binding to antigen 2 is fused to the N-terminus of HC of Fab that binds to antigen 1. The construct contains wild-type Fc.

FIG. 35 shows binding of TriNKETs which include a CAIX-binding domain and a NKG2D-binding domain to NKG2D expressed on EL4 cells.

FIG. 36 shows binding of TriNKETs that include a CAIX-binding domain and a NKG2D-binding domain, to CAIX expressed on human renal cell carcinoma cells A498.

FIG. 37A shows TriNKETs-mediated cytotoxicity of KHYG-1 effector cells towards CAIX-positive renal cell carcinoma A498 cells. The dotted line indicates the background killing of A498 cells during co-culture with rested KHYG-1 cells without addition of TriNKETs. FIG. 37B shows TriNKETs-mediated cytotoxicity of rested NK cells towards CAIX-positive renal cell carcinoma A498 cells.

### DETAILED DESCRIPTION

The invention provides multi-specific binding proteins that bind CAIX, ANO1, mesothelin, TROP2, CEA or Claudin-18.2 on a cancer cell and the NKG2D receptor and CD16 receptor on natural killer cells to activate the natural killer cells, pharmaceutical compositions comprising such multi-specific binding proteins, and therapeutic methods using such multi-specific proteins and pharmaceutical compositions, including for the treatment of cancer. Various aspects of the invention are set forth below in sections; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate. As used herein, the term “antigen-binding site” refers to the part of the immunoglobulin molecule that participates in antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

The term “tumor associated antigen” as used herein means any antigen including but not limited to a protein, glycoprotein, ganglioside, carbohydrate, lipid that is associated with cancer. Such antigen can be expressed on malignant cells or in the tumor microenvironment such as on tumor-associated blood vessels, extracellular matrix, mesenchymal stroma, or immune infiltrates.

As used herein, the terms “subject” and “patient” refer to an organism to be treated by the methods and compositions described herein. Such organisms preferably include, but are not limited to, mammals (e.g., marines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Proteins

The invention provides multi-specific binding proteins that bind CAIX, ANO1, mesothelin, TROP2, CEA or Claudin-18.2 on a cancer cell and the NKG2D receptor and CD16 receptor on natural killer cells to activate the natural killer cell. The multi-specific binding proteins are useful in the pharmaceutical compositions and therapeutic methods described herein. Binding of the multi-specific binding protein to the NKG2D receptor and CD16 receptor on natural killer cell enhances the activity of the natural killer cell toward destruction of a cancer cell. Binding of the multi-specific binding protein to CAIX, ANO1, mesothelin, TROP2, CEA or Claudin-18.2 on a cancer cell brings the cancer cell into proximity with the natural killer cell, which facilitates direct and indirect destruction of the cancer cell by the natural killer cell. Further description of exemplary multi-specific binding proteins is provided below.

The first component of the multi-specific binding proteins binds to NKG2D receptor-expressing cells, which can include but are not limited to NK cells, γδ T cells and CD8⁺ αβ T cells. Upon NKG2D binding, the multi-specific binding proteins may block natural ligands, such as ULBP6 and MICA, from binding to NKG2D and activating NKG2D receptors.

The second component of the multi-specific binding proteins binds to CAIX, ANO1, mesothelin, TROP2, CEA or Claudin-18.2-expressing cells. CAIX-expressing cells include, but are not limited to, renal cell carcinoma, breast cancer, glioblastoma, head and neck cancer, gastric cancers, bladder cancer, ovarian cancer, tumors of the esophagus, lung, colon, kidney, cervix and non-small cell lung carcinoma. Mesothelin-expressing cells include, but are not limited to, mesothelioma, ovarian cancer, pancreatic cancer, non-small cell lung cancer, breast cancer, cholangiocarcinoma, gastric cancer, uterine serous carcinoma, thymic carcinoma, and acute myeloid leukemia. ANO1-expressing cells include, but are not limited to, esophageal squamous cell cancer (ESCC), gastrointestinal stromal tumor (GIST), head and neck squamous cell carcinoma (HNSCC), pancreatic cancer, breast cancer, prostate cancer, and sarcoma. TROP2-expressing cells include, but are not limited to, breast, lung, gastric, colorectal, pancreatic, prostatic, cervical, head-and-neck cancer, nasopharyngeal carcinoma, and ovarian carcinoma. CEA-expressing cells include, but are not limited to, gastrointestinal cancer, colorectal cancer, pancreatic cancer, non-small cell lung cancer and breast cancer. Claudin-18.2-expressing cells include, but are not limited to, esophageal cancer, non-small cell lung carcinoma, ovarian cancer, colon cancer, and several forms of biliary ductal carcinoma.

The third component for the multi-specific binding proteins binds to cells expressing CD16, an Fc receptor on the surface of leukocytes including natural killer cells, macrophages, neutrophils, eosinophils, mast cells, and follicular dendritic cells.

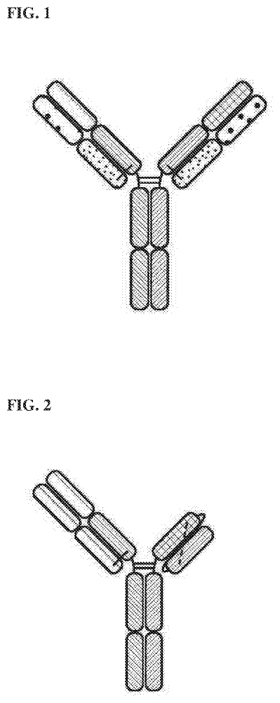

The multi-specific binding proteins described herein can take various formats. For example, one format is a heterodimeric, multi-specific antibody including a first immunoglobulin heavy chain, a first immunoglobulin light chain, a second immunoglobulin heavy chain and a second immunoglobulin light chain (FIG. 1). The first immunoglobulin heavy chain includes a first Fc (hinge-CH2-CH3) domain, a first heavy chain variable domain and optionally a first CH1 heavy chain domain. The first immunoglobulin light chain includes a first light chain variable domain and a first light chain constant domain. The first immunoglobulin light chain, together with the first immunoglobulin heavy chain, forms an antigen-binding site that binds NKG2D. The second immunoglobulin heavy chain comprises a second Fc (hinge-CH2-CH3) domain, a second heavy chain variable domain and optionally a second CH1 heavy chain domain. The second immunoglobulin light chain includes a second light chain variable domain and a second light chain constant domain. The second immunoglobulin light chain, together with the second immunoglobulin heavy chain, forms an antigen-binding site that binds CAIX, ANO1, mesothelin, TROP2, CEA or Claudin-18.2. The first Fc domain and second Fc domain together are able to bind to CD16 (FIG. 1). In some embodiments, the first immunoglobulin light chain is identical to the second immunoglobulin light chain.

Another exemplary format involves a heterodimeric, multi-specific antibody including a first immunoglobulin heavy chain, a second immunoglobulin heavy chain and an immunoglobulin light chain (FIG. 2). The first immunoglobulin heavy chain includes a first Fc (hinge-CH2-CH3) domain fused via either a linker or an antibody hinge to a single-chain variable fragment (scFv) composed of a heavy chain variable domain and light chain variable domain which pair and bind NKG2D, or bind one of the tumor associated antigen selected from CAIX, ANO1, mesothelin, TROP2, CEA and Claudin-18.2. The second immunoglobulin heavy chain includes a second Fc (hinge-CH2-CH3) domain, a second heavy chain variable domain and optionally a CH1 heavy chain domain. The immunoglobulin light chain includes a light chain variable domain and a light chain constant domain. The second immunoglobulin heavy chain pairs with the immunoglobulin light chain and binds to NKG2D or binds one of the tumor associated antigen selected from CAIX, ANO1, mesothelin, TROP2, CEA and Claudin-18.2. The first Fc domain and the second Fc domain together are able to bind to CD16 (FIG. 2).

One or more additional binding motifs may be fused to the C-terminus of the constant region CH3 domain, optionally via a linker sequence. In certain embodiments, the antigen-binding site could be a single-chain or disulfide-stabilized variable region (scFv) or could form a tetravalent or trivalent molecule.

In some embodiments, the multi-specific binding protein is in the Triomab form, which is a trifunctional, bispecific antibody that maintains an IgG-like shape. This chimera consists of two half antibodies, each with one light and one heavy chain, that originate from two parental antibodies.

In some embodiments, the multi-specific binding protein is the KiH Common Light Chain (LC) form, which involves the knobs-into-holes (KIHs) technology. The KIH involves engineering C_{H}3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structured-guided phage library screening (Atwell S, Ridgway J B, Wells J A, Carter P., Stable heterodimers from remodeling the domain interface of a homodimer using a phage display library, J. Mol. Biol. (1997) 270(1):26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426(9):1947-57; Mimoto F, Kadono S, Katada H, Igawa T, Kamikawa T, Hattori K. Crystal structure of a novel asymmetrically engineered Fc variant with improved affinity for FcgammaRs Mol. Immunol. (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the multi-specific binding protein is in the dual-variable domain immunoglobulin (DVD-Ig™) form, which combines the target binding domains of two monoclonal antibodies via flexible naturally occurring linkers, and yields a tetravalent IgG-like molecule.

In some embodiments, the multi-specific binding protein is in the Orthogonal Fab interface (Ortho-Fab) form. In the ortho-Fab IgG approach (Lewis S M, Wu X, Pustilnik A, Sereno A, Huang F, Rick H L, et al., Generation of bispecific IgG antibodies by structure-based design of an orthogonal Fab interface. Nat. Biotechnol. (2014) 32(2):191-8), structure-based regional design introduces complementary mutations at the LC and HC_{VH-CH1} interface in only one Fab, without any changes being made to the other Fab.

In some embodiments, the multi-specific binding protein is in the 2-in-1 Ig format. In some embodiments, the multi-specific binding protein is in the ES form, which is a heterodimeric construct containing two different Fabs binding to targets 1 and target 2 fused to the Fc. Heterodimerization is ensured by electrostatic steering mutations in the Fc.

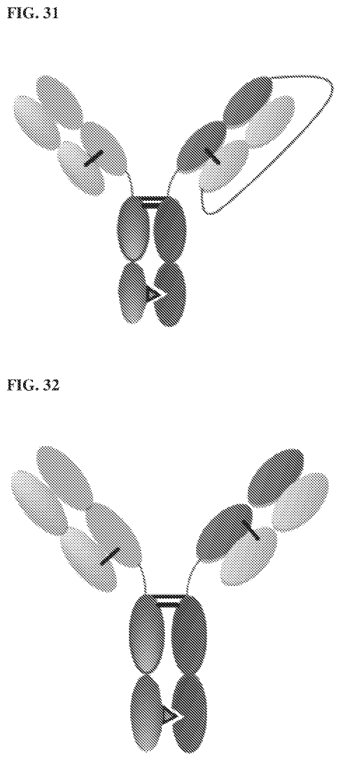

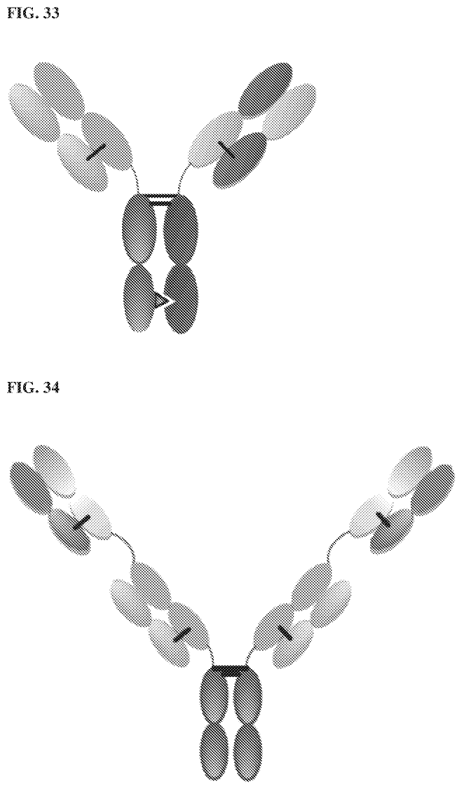

In some embodiments, the multi-specific binding protein is in the κλ-Body form, which is a heterodimeric construct with two different Fabs fused to Fc stabilized by heterodimerization mutations: Fab1 targeting antigen 1 contains kappa LC, while second Fab targeting antigen 2 contains lambda LC. FIG. 30A is an exemplary representation of one form of a κλ-Body; FIG. 30B is an exemplary representation of another κλ-Body.

In some embodiments, the multi-specific binding protein is in Fab Arm Exchange form (antibodies that exchange Fab arms by swapping a heavy chain and attached light chain (half-molecule) with a heavy-light chain pair from another molecule, which results in bispecific antibodies).

In some embodiments, the multi-specific binding protein is in the SEED Body form. The strand-exchange engineered domain (SEED) platform was designed to generate asymmetric and bispecific antibody-like molecules, a capability that expands therapeutic applications of natural antibodies. This protein engineered platform is based on exchanging structurally related sequences of immunoglobulin within the conserved CH3 domains. The SEED design allows efficient generation of AG/GA heterodimers, while disfavoring homodimerization of AG and GA SEED CH3 domains. (Muda M. et al., Protein Eng. Des. Sel. (2011, 24(5):447-54)).

In some embodiments, the multi-specific binding protein is in the LuZ-Y form, in which a leucine zipper is used to induce heterodimerization of two different HCs. (Wranik, B J. et al., J. Biol. Chem. (2012), 287:43331-9).

In some embodiments, the multi-specific binding protein is in the Cov-X-Body form. In bispecific CovX-Bodies, two different peptides are joined together using a branched azetidinone linker and fused to the scaffold antibody under mild conditions in a site-specific manner Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V R et al., PNAS (2010), 107(52); 22611-22616).

In some embodiments, the multi-specific binding protein is in an Oasc-Fab heterodimeric form that includes Fab binding to target 1, and scFab binding to target 2 fused to Fc. Heterodimerization is ensured by mutations in the Fc.

In some embodiments, the multi-specific binding protein is in a DuetMab form, which is an heterodimeric construct containing two different Fabs binding to antigens 1 and 2, and Fc stabilized by heterodimerization mutations. Fab1 and 2 contain differential S—S bridges that ensure correct LC and HC pairing.

In some embodiments, the multi-specific binding protein is in a CrossmAb form, which is an heterodimeric construct with two different Fabs binding to targets 1 and 2, fused to Fc stabilized by heterodimerization. CL and CH1 domains and VH and VL domains are switched, e.g., CH1 is fused in-line with VL, while CL is fused in-line with VH.

In some embodiments, the multi-specific binding protein is in a Fit-Ig form, which is a homodimeric construct where Fab binding to antigen 2 is fused to the N terminus of HC of Fab that binds to antigen 1. The construct contains wild-type Fc.

Table 1 lists peptide sequences of heavy chain variable domains and light chain variable domains that, in combination, can bind to NKG2D. The NKG2D-binding domains can vary in their binding affinities to NKG2D, nevertheless, they all activate human NKG2D and

NK cells.

TABLE 1

| | Heavy chain variable region | Light chain variable |
|---|---|---|
| Clones | amino acid sequence | region amino acid |
| ADI- | QVQLQQWGAGLLKPSETLSLTCAV | DIQMTQSPSTLSASVGDRVTITCR |
| 27705 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYNSYPI |
| | RGPWSFDPWGQGTLVTVSS | TFGGGTKVEIK |
| | (SEQ ID NO: 1) | (SEQ ID NO: 2) |
| | CDR1 (SEQ ID NO: 51)- | |
| | GSFSGYYWS | |
| | CDR2 (SEQ ID NO: 52)- | |
| | EIDHSGSTNYNPSLKS | |
| | CDR3 (SEQ ID NO: 53)- | |
| | ARARGPWSFDP | |
| ADI- | QVQLQQWGAGLLKPSETLSLTCAV | EIVLTQSPGTLSLSPGERATLSCRA |
| 27724 | YGGSFSGYYWSWIRQPPGKGLEWI | SQSVSSSYLAWYQQKPGQAPRLL |
| | GEIDHSGSTNYNPSLKSRVTISVDTS | IYGASSRATGIPDRFSGSGSGTDFT |
| | KNQFSLKLSSVTAADTAVYYCARA | LTISRLEPEDFAVYYCQQYGSSPIT |
| | RGPWSFDPWGQGTLVTVSS | FGGGTKVEIK |
| | (SEQ ID NO: 3) | (SEQ ID NO: 4) |
| ADI- | QVQLQQWGAGLLKPSETLSLTCAV | DIQMTQSPSTLSASVGDRVTITCR |
| 27740 | YGGSFSGYYWSWIRQPPGKGLEWI | ASQSIGSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYHSFYT |
| | RGPWSFDPWGQGTLVTVSS | FGGGTKVEIK |
| | (SEQ ID NO: 5) | (SEQ ID NO: 6) |
| ADI- | QVQLQQWGAGLLKPSETLSLTCAV | DIQMTQSPSTLSASVGDRVTITCR |
| 27741 | YGGSFSGYYWSWIRQPPGKGLEWI | ASQSIGSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQSNSYYT |
| | RGPWSFDPWGQGTLVTVSS | FGGGTKVEIK |
| | (SEQ ID NO: 7) | (SEQ ID NO: 8) |
| ADI- | QVQLQQWGAGLLKPSETLSLTCAV | DIQMTQSPSTLSASVGDRVTITCR |
| 27743 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYNSYPT |
| | RGPWSFDPWGQGTLVTVSS | FGGGTKVEIK |
| | (SEQ ID NO: 9) | (SEQ ID NO: 10) |
| ADI- | QVQLQQWGAGLLKPSETLSLTCAV | ELQMTQSPSSLSASVGDRVTITCR |
| 28153 | YGGSFSGYYWSWIRQPPGKGLEWI | TSQSISSYLNWYQQKPGQPPKLLI |
| | GEIDHSGSTNYNPSLKSRVTISVDTS | YWASTRESGVPDRFSGSGSGTDF |
| | KNQFSLKLSSVTAADTAVYYCARA | TLTISSLQPEDSATYYCQQSYDIPY |
| | RGPWGFDPWGQGTLVTVSS | TFGQGTKLEIK |
| | (SEQ ID NO: 11) | (SEQ ID NO: 12) |
| ADI- | QVQLQQWGAGLLKPSETLSLTCAV | DIQMTQSPSTLSASVGDRVTITCR |
| 28226 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYGSFPIT |
| | RGPWSFDPWGQGTLVTVSS | FGGGTKVEIK |
| | (SEQ ID NO: 13) | (SEQ ID NO: 14) |
| ADI- | QVQLQQWGAGLLKPSETLSLTCAV | DIQMTQSPSTLSASVGDRVTITCR |
| 28154 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTDFT |
| | KNQFSLKLSSVTAADTAVYYCARA | LTISSLQPDDFATYYCQQSKEVPW |
| | RGPWSFDPWGQGTLVTVSS | TFGQGTKVEIK |
| | (SEQ ID NO: 15) | (SEQ ID NO: 16) |
| ADI- | QVQLQQWGAGLLKPSETLSLTCAV | DIQMTQSPSTLSASVGDRVTITCR |
| 29399 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYNSFPT |
| | RGPWSFDPWGQGTLVTVSS | FGGGTKVEIK |
| | (SEQ ID NO: 17) | (SEQ ID NO: 18) |
| ADI- | QVQLQQWGAGLLKPSETLSLTCAV | DIQMTQSPSTLSASVGDRVTITCR |
| 29401 | YGGSFSGYYWSWIRQPPGKGLEWI | ASQSIGSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYDIYPT |
| | RGPWSFDPWGQGTLVTVSS | FGGGTKVEIK |
| | (SEQ ID NO: 19) | (SEQ ID NO: 20) |
| ADI- | QVQLQQWGAGLLKPSETLSLTCAV | DIQMTQSPSTLSASVGDRVTITCR |
| 29403 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYDSYPT |
| | RGPWSFDPWGQGTLVTVSS | FGGGTKVEIK |
| | (SEQ ID NO: 21) | (SEQ ID NO: 22) |
| ADI- | QVQLQQWGAGLLKPSETLSLTCAV | DIQMTQSPSTLSASVGDRVTITCR |
| 29405 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYGSFPT |
| | RGPWSFDPWGQGTLVTVSS | FGGGTKVEIK |
| | (SEQ ID NO: 23) | (SEQ ID NO: 24) |
| ADI- | QVQLQQWGAGLLKPSETLSLTCAV | DIQMTQSPSTLSASVGDRVTITCR |
| 29407 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYQSFPT |
| | RGPWSFDPWGQGTLVTVSS | FGGGTKVEIK |
| | (SEQ ID NO: 25) | (SEQ ID NO: 26) |
| ADI- | QVQLQQWGAGLLKPSETLSLTCAV | DIQMTQSPSTLSASVGDRVTITCR |
| 29419 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYSSFSTF |
| | RGPWSFDPWGQGTLVTVSS | GGGTKVEIK |
| | (SEQ ID NO: 27) | (SEQ ID NO: 28) |
| ADI- | QVQLQQWGAGLLKPSETLSLTCAV | DIQMTQSPSTLSASVGDRVTITCR |
| 29421 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYESYST |
| | RGPWSFDPWGQGTLVTVSS | FGGGTKVEIK |
| | (SEQ ID NO: 29) | (SEQ ID NO: 30) |
| ADI- | QVQLQQWGAGLLKPSETLSLTCAV | DIQMTQSPSTLSASVGDRVTITCR |
| 29424 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYDSFITF |
| | RGPWSFDPWGQGTLVTVSS | GGGTKVEIK |
| | (SEQ ID NO: 31) | (SEQ ID NO: 32) |
| ADI- | QVQLQQWGAGLLKPSETLSLTCAV | DIQMTQSPSTLSASVGDRVTITCR |
| 29425 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYQSYPT |
| | RGPWSFDPWGQGTLVTVSS | FGGGTKVEIK |
| | (SEQ ID NO: 33) | (SEQ ID NO: 34) |
| ADI- | QVQLQQWGAGLLKPSETLSLTCAV | DIQMTQSPSTLSASVGDRVTITCR |
| 29426 | YGGSFSGYYWSWIRQPPGKGLEWI | ASQSIGSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYHSFPT |
| | RGPWSFDPWGQGTLVTVSS | FGGGTKVEIK |
| | (SEQ ID NO: 35) | (SEQ ID NO: 36) |
| ADI- | QVQLQQWGAGLLKPSETLSLTCAV | DIQMTQSPSTLSASVGDRVTITCR |
| 29429 | YGGSFSGYYWSWIRQPPGKGLEWI | ASQSIGSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYELYSY |
| | RGPWSFDPWGQGTLVTVSS | TFGGGTKVEIK |
| | (SEQ ID NO: 37) | (SEQ ID NO: 38) |
| ADI- | QVQLQQWGAGLLKPSETLSLTCAV | DIQMTQSPSTLSASVGDRVTITCR |
| 29447 | YGGSFSGYYWSWIRQPPGKGLEWI | ASQSISSWLAWYQQKPGKAPKLL |
| | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYDTFITF |
| | RGPWSFDPWGQGTLVTVSS | GGGTKVEIK |
| | (SEQ ID NO: 39) | (SEQ ID NO: 40) |
| ADI- | QVQLVQSGAEVKKPGSSVKVSCKA | DIVMTQSPDSLAVSLGERATINCK |
| 27727 | SGGTFSSYAISWVRQAPGQGLEWM | SSQSVLYSSNNKNYLAWYQQKP |
| | GGIIPIFGTANYAQKFQGRVTITADE | GQPPKLLIYWASTRESGVPDRFSG |
| | STSTAYMELSSLRSEDTAVYYCAR | SGSGTDFTLTISSLQAEDVAVYYC |
| | GDSSIRHAYYYYGMDVWGQGTTV | QQYYSTPITFGGGTKVEIK |
| | TVSS | (SEQ ID NO: 42) |
| | (SEQ ID NO: 41) | CDR1 (SEQ ID NO: 57)- |
| | CDR1 (SEQ ID NO: 54)- | KSSQSVLYSSNNKNYLA |
| | GTFSSYAIS | CDR2 (SEQ ID NO: 58)- |
| | CDR2 (SEQ ID NO: 55)- | WASTRES |
| | GIIPIFGTANYAQKFQG | CDR3 (SEQ ID NO: 59)- |
| | CDR3 (SEQ ID NO: 56)- | QQYYSTPIT |
| | ARGDSSIRHAYYYYGMDV | |
| ADI- | QLQLQESGPGLVKPSETLSLTCTVS | EIVLTQSPATLSLSPGERATLSCRA |
| 29443 | GGSISSSSYYWGWIRQPPGKGLEWI | SQSVSRYLAWYQQKPGQAPRLLI |
| | GSIYYSGSTYYNPSLKSRVTISVDTS | YDASNRATGIPARFSGSGSGTDFT |
| | KNQFSLKLSSVTAADTAVYYCARG | LTISSLEPEDFAVYYCQQFDTWPP |
| | SDRFHPYPDYWGQGTLVTVSS | TFGGGTKVEIK |
| | (SEQ ID NO: 43) | (SEQ ID NO: 44) |
| | CDR1 (SEQ ID NO: 60)- | CDR1 (SEQ ID NO: 63)- |
| | GSISSSSYYWG | RASQSVSRYLA |
| | CDR2 (SEQ ID NO: 61)- | CDR2 (SEQ ID NO: 64)- |
| | SIYYSGSTYYNPSLKS | DASNRAT |
| | CDR3 (SEQ ID NO: 62)- | CDR3 (SEQ ID NO: 65)- |
| | ARGSDRFHPYFDY | QQFDTWPPT |
| ADI- | QVQLVQSGAEVKKPGSSVKVSCKA | DIVMTQSPDSLAVSLGERATINCE |
| 28200 | SGGTFSSYAISWVRQAPGQGLEWM | SSQSLLNSGNQKNYLTWYQQKPG |
| | GGIIPIFGTANYAQKFQGRVTITADE | QPPKPLIYWASTRESGVPDRFSGS |
| | STSTAYMELSSLRSEDTAVYYCAR | GSGTDFTLTISSLQAEDVAVYYCQ |
| | RGRKASGSFYYYYGMDVWGQGTT | NDYSYPYTFGQGTKLEIK |
| | VTVSS | (SEQ ID NO: 46) |
| | (SEQ ID NO: 45) | CDR1 (SEQ ID NO: 69)- |
| | CDR1 (SEQ ID NO: 66)- | ESSQSLLNSGNQKNYLT |
| | GTFSSYAIS | CDR2 (SEQ ID NO: 70)- |
| | CDR2 (SEQ ID NO: 67)- | WASTRES |
| | GIIPIFGTANYAQKFQG | CDR3 (SEQ ID NO: 71)- |
| | CDR3 (SEQ ID NO: 68)- | QNDYSYPYT |
| | ARRGRKASGSFYYYYGMDV | |
| ADI-27744 | EVQLLESGGGLVQPGGSLRLSCAA | DIQMTQSPSSVSASVGDRVTITCR |
| (A44) | SGFTFSSYAMSWVRQAPGKGLEW | ASQGIDSWLAWYQQKPGKAPKL |
| | VSAISGSGGSTYYADSVKGRFTISR | LIYAASSLQSGVPSRFSGSGSGTD |
| | DNSKNTLYLQMNSLRAEDTAVYY | FTLTISSLQPEDFATYYCQQGVSY |
| | CAKDGGYYDSGAGDYWGQGTLV | PRTFGGGTKVEIK |
| | TVSS | (SEQ ID NO: 133) |
| | (SEQ ID NO: 132) | CDR1 (SEQ ID NO:137)- |
| | CDR1 (SEQ ID NO: 134)- | RASQGIDSWLA |
| | FTFSSYAMS | CDR2 (SEQ ID NO:138)- |
| | CDR2 (SEQ ID NO: 135)- | AASSLQS |
| | AISGSGGSTYYADSVKG | CDR3 (SEQ ID NO:139)- |
| | CDR3 (SEQ ID NO: 136)- | QQGVSYPRT |
| | AKDGGYYDSGAGDY | |
| ADI- | EVQLVESGGGLVKPGGSLRLSCAA | DIQMTQSPSSVSASVGDRVTITCR |
| 27749 | SGFTFSSYSMNWVRQAPGKGLEW | ASQGISSWLAWYQQKPGKAPKLL |
| (A49) | VSSISSSSSYIYYADSVKGRFTISRD | IYAASSLQSGVPSRFSGSGSGTDF |
| | NAKNSLYLQMNSLRAEDTAVYYC | TLTISSLQPEDFATYYCQQGVSFP |
| | ARGAPMGAAAGWFDPWGQGTLV | RTFGGGTKVEIK |
| | TVSS | (SEQ ID NO: 141) |
| | (SEQ ID NO: 140) | CDR1 (SEQ ID NO: 145)- |
| | CDR1 (SEQ ID NO: 142)- | RASQGISSWLA |
| | FTFSSYSMN | CDR2 (SEQ ID NO: 146)- |
| | CDR2 (SEQ ID NO: 143)- | AASSLQS |
| | SISSSSSYIYYADSVKG | CDR3 (SEQ ID NO: 147)- |
| | CDR3 (SEQ ID NO: 144)- | QQGVSFPRT |
| | ARGAPMGAAAGWFDP | |
| ADI- | QVQLVQSGAEVKKPGASVKVSCK | EIVLTQSPGTLSLSPGERATLSCRA |
| 29463 | ASGYTFTGYYMHWVRQAPGQGLE | SQSVSSNLAWYQQKPGQAPRLLI |
| (F63) | WMGWINPNSGGTNYAQKFQGRVT | YGASTRATGIPARFSGSGSGTEFT |
| | MTRDTSISTAYMELSRLRSDDTAV | LTISSLQSEDFAVYYCQQDDYWP |
| | YYCARDTGEYYDTDDHGMDVWG | PTFGGGTKVEIK |
| | QGTTVTVSS | (SEQ ID NO: 149) |
| | (SEQ ID NO: 148) | CDR1 (SEQ ID NO: 153)- |
| | CDR1 (SEQ ID NO: 150)- | RASQSVSSNLA |
| | YTFTGYYMH | CDR2 (SEQ ID NO: 154)- |
| | CDR2 (SEQ ID NO: 151)- | GASTRAT |
| | WINPNSGGTNYAQKFQG | CDR3 (SEQ ID NO: 155)- |
| | CDR3 (SEQ ID NO: 152)- | QQDDYWPPT |
| | ARDTGEYYDTDDHGMDV | |
| ADI-29379 | QVQLVQSGAEVKKPGASVKVSCK | EIVMTQSPATLSVSPGERATLSCR |
| (E79) | ASGYTFTSYYMHWVRQAPGQGLE | ASQSVSSNLAWYQQKPGQAPRLL |
| | WMGIINPSGGSTSYAQKFQGRVTM | IYGASTRATGIPARFSGSGSGTEFT |
| | TRDTSTSTVYMELSSLRSEDTAVY | LTISSLQSEDFAVYYCQQYDDWP |
| | YCARGAPNYGDTTHDYYYMDVW | FTFGGGTKVEIK |
| | GKGTTVTVSS | (SEQ ID NO: 157) |
| | (SEQ ID NO: 156) | CDR1 (SEQ ID NO: 161)- |
| | CDR1 (SEQ ID NO: 158)- | RASQSVSSNLA |
| | YTFTSYYMH | CDR2 (SEQ ID NO: 162)- |
| | CDR2 (SEQ ID NO: 159)- | GASTRAT |
| | IINPSGGSTSYAQKFQG | CDR3 (SEQ ID NO: 163)- |
| | CDR3 (SEQ ID NO: 160)- | QQYDDWPFT |
| | ARGAPNYGDTTHDYYYMDV | |
| ADI- | QVQLQQWGAGLLKPSETLSLTCAV | DIQMTQSPSTLSASVGDRVTITCR |
| 29447 | YGGSFSGYYWSWIRQPPGKGLEWI | ASQSISSWLAWYQQKPGKAPKLL |
| (F47) | GEIDHSGSTNYNPSLKSRVTISVDTS | IYKASSLESGVPSRFSGSGSGTEFT |
| | KNQFSLKLSSVTAADTAVYYCARA | LTISSLQPDDFATYYCQQYDTFITF |
| | RGPWSFDPWGQGTLVTVSS | GGGTKVEIK |
| | (SEQ ID NO: 164) | (SEQ ID NO: 165) |
| | CDR1 (SEQ ID NO: 166)- | CDR1 (SEQ ID NO: 169)- |
| | GSFSGYYWS | RASQSISSWLA |
| | CDR2 (SEQ ID NO: 167)- | CDR2 (SEQ ID NO: 170)- |
| | EIDHSGSTNYNPSLKS | KASSLES |
| | CDR3 (SEQ ID NO: 168)- | CDR3 (SEQ ID NO: 171)- |
| | ARARGPWSFDP | QQYDTFIT |

Alternatively, a heavy chain variable domain defined by SEQ ID NO:47 can be paired with a light chain variable domain defined by SEQ ID NO:48 to form an antigen-binding site that can bind to NKG2D, as illustrated in U.S. Pat. No. 9,273,136.

SEQ ID NO: 47
QVQLVESGGGLVKPGGSLRLSCAASGFTFSSYGMHW
VRQAPGKGLEWVAFIRYDGSNKYYADSVKGRFTISR
DNSKNTLYLQMNSLRAEDTAVYYCAKDRGLGDGTYF
DYWGQGTTVTVSS
SEQ ID NO: 48
QSALTQPASVSGSPGQSITISCSGSSSNIGNNAVNW
YQQLPGKAPKLLIYYDDLLPSGVSDRFSGSKSGTSA
FLAISGLQSEDEADYYCAAWDDSLNGPVFGGGTKLT
VL

Alternatively, a heavy chain variable domain defined by SEQ ID NO:49 can be paired with a light chain variable domain defined by SEQ ID NO:50 to form an antigen-binding site that can bind to NKG2D, as illustrated in U.S. Pat. No. 7,879,985.

SEQ ID NO: 49
QVHLQESGPGLVKPSETLSLTCTVSDDSISSYYWSWI
RQPPGKGLEWIGHISYSGSANYNPSLKSRVTISVDTS
KNQFSLKLSSVTAADTAVYYCANWDDAFNIWGQGTMV
TVSS
SEQ ID NO: 50
EIVLTQSPGTLSLSPGERATLSCRASQSVSSSYLAWY
QQKPGQAPRLLIYGASSRATGIPDRFSGSGSGTDFTL
TISRLEPEDFAVYYCQQYGSSPWTFGQGTKVEIK

Table 2 lists peptide sequences of heavy chain variable domains and light chain variable domains that, in combination, can bind to CAIX.

TABLE 2

| | Heavy | Light |
|---|---|---|
| | chain | chain |
| | variable | variable |
| | domain | domain |
| | amino | amino |
| | acid | acid |
| Clones | sequence | sequence |
| Girentuximab | DVKLVESGG | DIVMTQSQ |
| (PCT/EP2002/ | GLVKLGGSL | RFMSTTVG |
| 001283) | KLSCAASGF | DRVSITCK |
| | TFSNYYMSW | ASQNVVSA |
| | VRQTPEKRL | VAWYQQKP |
| | ELVAAINSD | GQSPKLLI |
| | GGITYYLDT | YSASNRYT |
| | VKGRFTISR | GVPDRFTG |
| | DNAKNTLYL | SGSGTDFT |
| | QMSSLKSED | LTISNMQS |
| | TALFYCARH | EDLADFFC |
| | RSGYFSMDY | QQYSNYPW |
| | WGQGTSVTV | TFGGGTKL |
| | SS | EIKR |
| | (SEQ ID | (SEQ ID |
| | NO: 72) | NO: 73) |
| | CDR1 | CDR1 |
| | (SEQ ID | (SEQ ID |
| | NO: 74)- | NO: 77)- |
| | GFTFSNY | QNVVSAVA |
| | CDR2 | CDR2 |
| | (SEQ ID | (SEQ ID |
| | NO: 75)- | NO: 78)- |
| | NSDGGI | SASNRYT |
| | CDR3 | CDR3 |
| | (SEQ ID | (SEQ ID |
| | NO: 76)- | NO: 79)- |
| | HRSGYF | QQYSNYPWT |
| | SMDY | |
| BAY79-4620 | QVELVESG | DIQMTQSPS |
| (U.S. | GGLVQPGG | SLSASVGDR |
| Pat. No. | SLRLSCAA | VTITCRASQ |
| 8,168,758) | SGFTFSSY | DINNYLSWY |
| | GMSWVRQA | QQKPGKAPK |
| | PGKGLEWV | LLIYGASNL |
| | SGISSLGS | QSGVPSRFS |
| | TTYYADSV | GSGSGTDFT |
| | KGRFTISR | LTISSLQPE |
| | DNSKNTLY | DFAVYYCQQ |
| | LQMNSLRA | YYGRPTTFG |
| | EDTAVYYC | QGTKVEIK |
| | ARTGSPGT | (SEQ ID |
| | FMHGDHWG | NO: 81) |
| | QGTLVTVS | CDR1 |
| | S | (SEQ ID |
| | (SEQ ID | NO: 85)- |
| | NO: 80) | RASQDI |
| | CDR1 | NNYLS |
| | (SEQ ID | CDR2 |
| | NO: 82)- | (SEQ ID |
| | GFTFS | NO: 86)- |
| | SYGMS | YGASNLQS |
| | CDR2 | CDR3 |
| | (SEQ ID | (SEQ ID |
| | NO: 83)- | NO: 87)- |
| | GISSLGST | QQYYGRPT |
| | TYYADSVK | |
| | G | |
| | CDR3 | |
| | (SEQ ID | |
| | NO: 84)- | |
| | TGSPGTFM | |
| | HGDH | |
| Anti-CAIX | EVQLVESGG | DIVMTQSPS |
| (U.S. | GLVQPGGSL | SLSASVGDR |
| Pat. No. | RLSCAASGF | VTITCRASQ |
| 12/016,884) | TFSNYYMSW | NVVSAVAWY |
| | VRQAPGQRL | LQKPGKSPK |
| | ELVSAINSD | LLIYSASNR |
| | GGITYYLDS | YTGVPDRFT |
| | VKG | GSGS |
| | RFTISRDN | GTDFTLTI |
| | AKNTLYLQ | SSLQPEDF |
| | MGSLRAED | ATYFCQQY |
| | MAVYYCAR | SNYPWTFG |
| | HRSGYFSM | GGTKLEIK |
| | DYWGQGTL | (SEQ ID |
| | VTVSS | NO: 89) |
| | (SEQ ID | CDR1 |
| | NO: 88) | (SEQ ID |
| | CDR1 | NO: 93)- |
| | (SEQ ID | QNVVSAVA |
| | NO: 90)- | CDR2 |
| | GFTFSNY | (SEQ ID |
| | CDR2 | NO:94)- |
| | (SEQ ID | SASNRYT |
| | NO: 91)- | CDR3 |
| | NSDGGI | (SEQ ID |
| | CDR3 | NO: 95)- |
| | (SEQ ID | QQYSN |
| | NO: 92)- | YPWT |
| | HRSGYFSMDY | |

Alternatively, novel antigen-binding sites that bind to CALX can be identified by screening for binding to the amino acid sequence defined by SEQ ID NO:96.

SEQ ID NO: 96
MAPLCPSPWLPLLIPAPAPGLTVQLLLSLLLLVPVHPQ
RLPRMQEDSPLGGGSSGEDDPLGEEDLPSEEDSPREED
PPGEEDLPGEEDLPGEEDLPEVKPKSEEEGSLKLEDLP
TVEAPGDPQEPQNNAHRDKEGDDQSHWRYGGDPPWPRV
SPACAGRFQSPVDIRPQLAAFCPALRPLELLGFQLPPL
PELRLRNNGHSVQLTLPPGLEMALGPGREYRALQLHLH
WGAAGRPGSEHTVEGHRFPAEIHVVHLSTAFARVDEAL
GRPGGLAVLAAFLEEGPEENSAYEQLLSRLEEIAEEGS
ETQVPGLDISALLPSDFSRYFQYEGSLTTPPCAQGVIW
TVFNQTVMLSAKQLHTLSDTLWGPGDSRLQLNFRATQP
LNGRVIEASFPAGVDSSPRAAEPVQLNSCLAAGDILAL
VFGLLFAVTSVAFLVQMRRQHRRGTKGGVSYRPAEVAE
TGA

Table 3 lists peptide sequences of heavy chain variable domains and light chain variable domains that, in combination, can bind to ANO1.

TABLE 3

| | Heavy chain | Light chain |
|---|---|---|
| | variable | variable |
| | domain amino | domain amino |
| Clones | acid sequence | acid sequence |
| ANO1 | QVQLKESGPGLV | DVVLTQTPPTLS |
| antibody | QPSQTLTLTCTV | ATIGQSVSISCR |
| (PCT/US20 | SGFSLSNYDMHW | SSQSLLHSNGNT |
| 12/067430) | VRQSPGKGLDWM | YLNWLLQRPGQP |
| | GVIWGNGKTQYN | PQLLIYLVSRLE |
| | SGLTSRLSISRD | SGVPNRFSGSGS |
| | TSKSQVFLKMNS | GTDFTLKISGVE |
| | LQTEDTAIYFCT | AEDLGVYYCVQS |
| | RSGYYYDGSYYS | THAPAFGGGTKL |
| | LFDYWGQG | ELK |
| | VMVTVSS | (SEQ ID |
| | (SEQ ID | NO: 98) |
| | NO: 97) | CDR1 |
| | CDR1 | (SEQ ID |
| | (SEQ ID | NO: 102)- |
| | NO: 99)- | RSSQSLLH |
| | NYDMH | SNGNTYLN |
| | CDR2 | CDR2 |
| | (SEQ ID | (SEQ ID |
| | NO: 100)- | NO: 103)- |
| | VIWGNGKT | LVSRLES |
| | QYNSGLTS | CDR3 |
| | CDR3 | (SEQ ID |
| | (SEQ ID | NO: 104)- |
| | NO: 101)- | VQSTHAPA |
| | SGYYYDGS | |
| | YYSLFDY | |

Alternatively, novel antigen-binding sites that bind to ANO1 can be identified by screening for binding to the amino acid sequence defined by SEQ ID NO:105.

SEQ ID NO: 105
MRVNEKYSTLPAEDRSVHIINICAIEDIGYLPSEGTLLNSL
SVDPDAECKYGLYFRDGRRKVDYILVYHHKRPSGNRTLVRR
VQHSDTPSGARSVKQDHPLPGKGASLDAGSGEPPMDYHEDD
KRFRREEYEGNLLEAGLELERDEDTKIHGVGFVKIHAPWNV
LCREAEFLKLKMPTKKMYHINETRGLLKKINSVLQKITDPI
QPKVAEHRPQTMKRLSYPFSREKQHLFDLSDKDSFFDSKTR
STIVYEILKRTTCTKAKYSMGITSLLANGVYAAAYPLHDGD
YNGENVEFNDRKLLYEEWARYGVFYKYQPIDLVRKYFGEKI
GLYFAWLGVYTQMLIPASIVGIIVFLYGCATMDENIPSMEM
CDQRHNITMCPLCDKTCSYWKMSSACATARASHLFDNPATV
FFSVFMALWAATFMEHWKRKQMRLNYRWDLTGFEEEEEAVK
DHPRAEYEARVLEKSLKKESRNKEKRRHIPEESTNKWKQRV
KTAMAGVKLTDKVKLTWRDRFPAYLTNLVSIIFMIAVTFAI
VLGVIIYRISMAAALAMNSSPSVRSNIRVTVTATAVIINLV
VIILLDEVYGCIARWLTKIEVPKTEKSFEERLIFKAFLLKF
VNSYTPIFYVAFFKGRFVGRPGDYVYIFRSFRMEECAPGGC
LMELCIQLSIIMLGKQLIQNNLFEIGIPKMKKLIRYLKLKQ
QSPPDHEECVKRKQRYEVDYNLEPFAGLTPEYMEMIIQFGF
VTLFVASFPLAPLFALLNNIIEIRLDAKKFVTELRRPVAVR
AKDIGIWYNILRGIGKLAVIINAFVISFTSDFIPRLVYLYM
YSKNGTMHGFVNHTLSSFNVSDFQNGTAPNDPLDLGYEVQI
CRYKDYREPPWSENKYDISKDFWAVLAARLAFVIVFQNLVM
FMSDFVDWVIPDIPKDISQQIHKEKVLMVELFMREEQDKQQ
LLETWMEKERQKDEPPCNHHNTKACPDSLGSPAPSHAYHGG
VL

Table 4 lists peptide sequences of heavy chain variable domains and light chain variable domains that, in combination, can bind to mesothelin.

TABLE 4

| | Heavy chain | Light chain |
|---|---|---|
| | variable | variable |
| | domain amino | domain amino |
| Clones | acid sequence | acid sequence |
| Amatuximab | QVQLQQSGPELE | DIELTQSPAIMS |
| (U.S. | KPGASVKISCKA | ASPGEKVTMTC |
| Pat. No. | SGYSFTGYTMNW | SASSSVSYMHW |
| 7,592,426) | VKQSHGKSLEWI | YQQKSGTSPKR |
| | GLITPYNGASSY | WIYDTSKLASG |
| | NQKFRGKATLTV | VPGRFSGSGSG |
| | DKSSSTAYMDLL | NSYSLTISSVE |
| | SLTSEDSAVYFC | AEDDATYYCQQ |
| | ARGGYDGRGFDY | WSKHPLTFGSG |
| | WGSGTPVTVSSA | TKVEIKR |
| | (SEQ ID | (SEQ ID |
| | NO: 106) | NO: 107) |
| | CDR1 | CDR1 |
| | (SEQ ID | (SEQ ID |
| | NO: 108)- | NO: 111)- |
| | GYSFTGY | SSVSYMH |
| | CDR2 | CDR2 |
| | (SEQ ID | (SEQ ID |
| | NO: 109)- | NO: 112)- |
| | TPYNGA | DTSKLAS |
| | CDR3 | CDR3 |
| | (SEQ ID | (SEQ ID |
| | NO: 110)- | NO: 113)- |
| | GGYDGRGFDY | QQWSKHPLT |
| Anti- | EVHLVESGGGLV | EIVLTQSPGTLS |
| mesothelin | QPGGSLRLSCA | LSPGERATLSCR |
| (U.S. | ASGFTFSRYWMS | ASQSVSSSYLAW |
| Pat. No. | WVRQAQGKGL | YQQKPGQAPR |
| 8,425,904) | EWVASIKQAGSE | LLIYGASSRATG |
| | KTYVDSVKGRF | IPDRFSGSGSGT |
| | TISRDNAKNSLS | DFTLTISRLEPE |
| | LQMNSLRAEDT | DFAVYYCQQYGS |
| | AVYYCAREGAYY | SQYTFGQGTKLE |
| | YDSASYYPYY | IK |
| | YYYSMDVWGQGT | (SEQ ID |
| | TVTVSS | NO: 115) |
| | (SEQ ID | CDR1 |
| | NO: 114) | (SEQ ID |
| | CDR1 | NO: 119)- |
| | (SEQ ID | QSVSSSYLA |
| | NO: 116)- | CDR2 |
| | GFTFSRY | (SEQ ID |
| | CDR2 | NO: 120)- |
| | (SEQ ID | GASSRAT |
| | NO: 117)- | CDR3 |
| | KQAGSE | (SEQ ID |
| | CDR3 | NO: 121)- |
| | (SEQ ID | QQYGSSQYT |
| | NO: 118)- | |
| | EGAYYYDSASY | |
| | YPYYYYYSMDV | |
| Anetumab | QVELVQSGAEV | DIALTQPASVSG |
| (U.S. | KKPGESLKISC | SPGQSITISCTG |
| Pat. No. | KGSGYSFTSYW | TSSDIGGYNSVS |
| 9,023,351) | IGWVRQAPGKG | WYQQHPGKAPKL |
| | LEWMGIIDPGD | MIYGVNNRPSGV |
| | SRTRYSPSFQG | SNRFSGSK |
| | QVTISADKSIS | SGNTASLTISGL |
| | TAYLQWSSLKA | QAEDEADYYCS |
| | SDTAM | |
| | YYCARGQLYG | SYDIESATPVF |
| | GTYMDGWGQGT | GGGTKLTVLG |
| | LVTVSSA | (SEQ ID |
| | (SEQ ID | NO: 123) |
| | NO: 122) | CDR1 |
| | CDR1 | (SEQ ID |
| | (SEQ ID | NO: 127)- |
| | NO: 124)- | SSDIGGYNSVS |
| | GYSFTSY | CDR2 |
| | CDR2 | (SEQ ID |
| | (SEQ ID | NO: 128)- |
| | NO: 125)- | GVNNRPS |
| | DPGDSR | CDR3 |
| | CDR3 | (SEQ ID |
| | (SEQ ID | NO: 129)- |
| | NO: 126)- | SSYDIESATPV |
| | GQLYGGTYMDG | |

Alternatively, novel antigen-binding sites that bind to mesothelin can be identified by screening for binding to the amino acid sequence defined by SEQ ID NO:130.

SEQ ID NO: 130
MALPTARPLLGSCGTPALGSLLFLLFSLGWVQPSRTLAGET
GQEAAPLDGVLANPPNISSLSPRQLLGFPCAEVSGLSTERV
RELAVALAQKNVKLSTEQLRCLAHRLSEPPEDLDALPLDLL
LFLNPDAFSGPQACTRFFSRITKANVDLLPRGAPERQRLLP
AALACWGVRGSLLSEADVRALGGLACDLPGRFVAESAEVLL
PRLVSCPGPLDQDQQEAARAALQGGGPPYGPPSTWSVSTMD
ALRGLLPVLGQPIIRSIPQGIVAAWRQRSSRDPSWRQPERT
ILRPRFRREVEKTACPSGKKAREIDESLIFYKKWELEACVD
AALLATQMDRVNAIPFTYEQLDVLKHKLDELYPQGYPESVI
QHLGYLFLKMSPEDIRKWNVTSLETLKALLEVNKGHEMSPQ
APRRPLPQVATLIDRFVKGRGQLDKDTLDTLTAFYPGYLCS
LSPEELSSVPPSSIWAVRPQDLDTCDPRQLDVLYPKARLAF
QNMNGSEYFVKIQSFLGGAPTEDLKALSQQNVSMDLATFMK
LRTDAVLPLTVAEVQKLLGPHVEGLKAEERHRPVRDWILRQ
RQDDLDTLGLGLQGGIPNGYLVLDLSMQEALSGTPCLLGPG
PVLTVLALLLASTLA

Table 5 lists peptide sequences of heavy chain variable domains and light chain variable domains that, in combination, can bind to TROP2.

TABLE 5

| | Heavy chain | Light chain |
|---|---|---|
| | variable | variable |
| | domain amino | domain amino |
| Clones | acid sequence | acid sequence |
| IMMU-132 | SVQLQQSGSEL | DIQLTQSPSSLSA |
| (U.S. | KKPGASVKVSC | SVGDRVSITCKA |
| Pat. No. | KASGYTFTNYG | SQDVSIAVAWYQ |
| 7,238,785) | MNWVKQAPGQG | QKPGKAPKLLIY |
| | LKWMGWINTYT | SASYRYTGVPDR |
| | GEPTYTDDFKGR | FSGSGSGTDFTL |
| | FAFSLDTSVST | TISSLQPEDFAV |
| | AYLQISSLKAD | YYCQQHYITPLTF |
| | DT | |
| | AVYFCARGGFGS | GAGTKVEIKR |
| | SYWYFDVWGQ | (SEQ ID |
| | GSLVTVSS | NO: 173) |
| | (SEQ ID | CDR1 |
| | NO: 172) | (SEQ ID |
| | CDR1 | NO: 177)- |
| | (SEQ ID | QDVSIAVA |
| | NO: 174)- | CDR2 |
| | GYTFTNY | (SEQ ID |
| | CDR2 | NO: 178)- |
| | (SEQ ID | SASYRYT |
| | NO: 175)- | CDR3 |
| | NTYTGE | (SEQ ID |
| | CDR3 | NO: 179)- |
| | (SEQ ID | QQHYITPLT |
| | NO: 176)- | |
| | GGFGSSYWYFDV | |
| Anti-TROP2 | QIQLVQSGHEVK | DIQMTQSPSSLSA |
| antibody | KPGASVKVSCK | SVGDRVTITCKA |
| (PCT/CA08/ | ASGYTFTNYGMN | SQDVSIAVAWYQ |
| 000979) | WVRQAPGQGL | QKPGKAPKVLI |
| | EWMGWINTKTGE | YSASYRYTGVPD |
| | PTYAEEFKGRF | RFSGSGSGTDFT |
| | VFSLETSASTAY | FTISSLQPEDIA |
| | LQISSLKAEDTA | VYYCQQHYITPL |
| | MYFCGRGGYGSS | TFGGGTKVEIK |
| | YWYFDVWGQ | (SEQ ID |
| | GTTVTVSS | NO: 181) |
| | (SEQ ID | CDR1 |
| | NO: 180) | (SEQ ID |
| | CDR1 | NO: 185)- |
| | (SEQ ID | QDVSIAVA |
| | NO: 182)- | CDR2 |
| | GYTFTNY | (SEQ ID |
| | CDR2 | NO: 186)- |
| | (SEQ ID | SASYRYT |
| | NO: 183)- | CDR3 |
| | NTKTGE | (SEQ ID |
| | CDR3 | NO: 187)- |
| | (SEQ ID | QQHYITPLT |
| | NO: 184)- | |
| | GGYGSSYWYFDV | |
| Anti-TROP2 | QVQLVQSGAEV | DIQMTQSPSSLS |
| antibody | KKPGASVKVSC | ASVGDRVTITCK |
| (U.S. | KASGYTFTTAG | ASQDVSTAVAWY |
| Pat. No. | MQWVRQAPGQG | QQKPGKAPKLLI |
| 15/187,179) | LEWMGWINTHS | YSASYRYTGVPS |
| | GVPKYAEDFKG | RFSGSGSGTDFT |
| | RVTISADTSTS | LTISSLQPEDFA |
| | TAYLQLSSLKS | VYYCQQHYITPL |
| | EDTAVYYCARS | TFGQGTKLEIKR |
| | GFGSSYWYFDV | T |
| | WGQGTLVTVSS | (SEQ ID |
| | (SEQ ID | NO: 189) |
| | NO: 188) | DIVMTQSPSSLS |
| | QVQLVQSGAEV | ASVGDRVTITCK |
| | KKPGASVKVSC | ASQDVSTAVAWY |
| | KASGYTFTTAG | QQKPGKAPKLLI |
| | MQWVRQAPGQG | YSASYRYTGVPS |
| | LEWMGWINTHS | RFSGSGSGTDFT |
| | GVPKYAEDFKG | LTISSLQPEDFA |
| | RVTISLDTSTS | VYYCQQHYITPL |
| | TAYLQLSSLKS | TFGQGTKLEIKR |
| | EDTAVYYCARS | T |
| | GFGSSYWYFDV | (SEQ ID |
| | WGQGTLVTVSS | NO: 191) |
| | (SEQ ID | DIVMTQSPSSLSA |
| | NO: 190) | SVGDRVTITCKA |
| | QIQLVQSGAEVK | SQDVSTAVAWYQ |
| | KPGESVKVSCK | QKPGKQPKLLIYS |
| | ASGYTFTTAGMQ | ASYRYTGVPSRFS |
| | WVQQMPGKGLEW | GSGSGTDFTLTIS |
| | MGWINTHSGVPK | SLQPEDFAVYYCQ |
| | YAEDFKGRVTFS | QHYITPLTFGQGT |
| | LDTSTSTAYLQL | KLEIKR |
| | SSLKSEDTAVYY | (SEQ ID |
| | CARSGFGSSYWY | NO: 193) |
| | FDVWGQGTLVTV | |
| | SS | |
| | (SEQ ID | |
| | NO: 192) | |

Alternatively, novel antigen-binding sites that bind to TROP2 can be identified by screening for binding to the amino acid sequence defined by SEQ ID NO:194.

SEQ ID NO: 194
MARGPGLAPPPLRLPLLLLVLAAVTGHTA
AQDNCTCPTNKMTVCSPDGPGGRCQCRAL
GS GMAVDCSTLTSKCLLLKARMSAPKNA
RTLVRPSEHALVDNDGLYDPDCDPEGRFK
ARQCNQTSVCWCVNSVGVRRTDKGDLSLR
CDELVRTHHILIDLRHRPTAGAFNHSDLD
AELRRLFRERYRLHPKFVAAVHYEQPTIQ
IELRQNTSQKAAGDVDIGDAAYYFERDIK
GESLFQGRGGLDLRVRGEPLQVERTLIYY
LDEIPPKFSMKRLTAGLIAVIVVVVVALV
AGMAVLVITNRRKSGKYKKVEIKELGELR
KEPSL

Table 6 lists peptide sequences of heavy chain variable domains and light chain variable domains that, in combination, can bind to CEA.

TABLE 6

| | Heavy chain | Light chain |
|---|---|---|
| | variable | variable |
| | domain amino | domain amino |
| Clones | acid sequence | acid sequence |
| labetuzumab | EVQLVESGGGWQ | DIQLTQSPSSLSA |
| | PGRSLRLSCSAS | SVGDRVTITCKAS |
| | GFDFTTYWMSWV | QDVGTSVAWYQQK |
| | RQAPGKGLEWI | PGKAPKLLIYWTS |
| | GEIHPDSSTINY | TRHTGVPSRFSGS |
| | APSLKDRFTISR | GSGTDFTFTISSL |
| | DNAKNTLFLQMD | QPEDIATYYCQQY |
| | SLRPEDTGVYFC | SLYRSFGQGTKVE |
| | ASLYFGFPWFA | IK |
| | YWGQGTPVTVSS | (SEQ ID |
| | (SEQ ID | NO: 196) |
| | NO: 195) | CDR1 |
| | CDR1 | (SEQ ID |
| | (SEQ ID | NO: 200)- |
| | NO: 197)- | |
| | GFDFTTY | |
| | CDR2 | QDVGTSVA |
| | (SEQ ID | CDR2 |
| | NO: 198)- | (SEQ ID |
| | HPDSST | NO: 201)- |
| | CDR3 | WTSTRHT |
| | (SEQ ID | CDR3 |
| | NO: 199)- | (SEQ ID |
| | LYFGFPWFAY | NO: 202)- |
| | | QQYSLYRS |
| cergutuzumab | QVQLVQSGAEVK | DIQMTQSPSSLSA |
| | KPGASVKVSCK | SVGDRVTITCKA |
| | ASGYTFTEFGMN | SAAVGTYVAWYQQ |
| | WVRQAPGQGLE | KPGKAPKLLIYSA |
| | WMGWINTKTGEA | SYRKRGVPSRFSG |
| | TYVEEFKGRVTF | SGSGTDFTLTISSL |
| | TTDTSTSTAYM | QPEDFATYYCHQYY |
| | ELRSLRSDDTAV | TYPLFTFGQGTKLE |
| | YYCARWDFAYYV | IKR |
| | EAMDYWGQGTT | (SEQ ID |
| | VTVSSA | NO: 204) |
| | (SEQ ID | CDR1 (SEQ ID |
| | NO: 203) | NO: 208)- |
| | CDR1 | AAVGTYVA |
| | (SEQ ID | CDR2 |
| | NO: 205- | (SEQ ID |
| | GYTFTEF | NO: 209)- |
| | CDR2 | SASYRKR |
| | (SEQ ID | CDR3 |
| | NO: 206)- | (SEQ ID |
| | NTKTGE | NO: 210)- |
| | CDR3 | HQYYTYPLFT |
| | (SEQ ID | |
| | NO: 207)- | |
| | WDFAYYVEAMDY | |
| anti-CEA | QVQLQQSGAELVR | DIQLTQSPAIMS |
| (U.S. | SGASIKLSCTAS | ASPGEKVTMTCS |
| Pat. No. | GFNIKHYYMHWV | ASSSVSYIHWYQ |
| 9,056,911) | KQRPEQGLEWI | QKSGTSPKRWV |
| | GWINPENVDTEY | YDTSKLASGVPA |
| | APKFQGKATMTA | RFSGSGSGTSYS |
| | DTSSNTAYLQLS | LTISTMEAEDAA |
| | SLTSEDTAVYYC | TYYCQQWNNNPY |
| | NHYRYAGGGALD | SFGGGTKVVIK |
| | YWGQGTTVTVSS | TV |
| | A | (SEQ ID |
| | (SEQ ID | NO: 212) |
| | NO: 211) | CDR1 |
| | CDR1 | (SEQ ID |
| | (SEQ ID | NO: 216)- |
| | NO: 213)- | SSVSYIH |
| | GFNIKHY | CDR2 |
| | CDR2 | (SEQ ID |
| | (SEQ ID | NO: 217)- |
| | NO: 214)- | DTSKLAS |
| | NPENVD | CDR3 |
| | CDR3 | (SEQ ID |
| | (SEQ ID | NO: 218)- |
| | NO: 215)- | QQWNNNPYS |
| | YRYAGGGALDY | |

Alternatively, novel antigen-binding sites that bind to CEA can be identified by screening for binding to the amino acid sequence defined by SEQ ID NO:219.

SEQ ID NO: 219
MESPSAPPHRWCIPWQRLLLTASLLTFWNPPTTAKLTIEST
PFNVAEGKEVLLLVHNLPQHLFGYSWYKGERVDGNRQIIGY
VIGTQQATPGPAYSGREIIYPNASLLIQNIIQNDTGFYTLH
VIKSDLVNEEATGQIRVYPELPKPSISSNNSKPVEDKDAVA
FTCEPETQDATYLWWVNNQSLPVSPRLQLSNGNRTLTLFNV
TRNDTASYKCETQNPVSARRSDSVILNVLYGPDAPTISPLN
TSYRSGENLNLSCHAASNPPAQYSWFVNGTFQQSTQELFIP
NITVNNSGSYTCQAHNSDTGLNRTTVTTITVYAEPPKPFIT
SNNSNPVEDEDAVALTCEPEIQNTTYLWWVNNQSLPVSPRL
QLSNDNRTLTLLSVTRNDVGPYECGIQNKLSVDHSDPVILN
VLYGPDDPTISPSYTYYRPGVNLSLSCHAASNPPAQYSWLI
DGNIQQHTQELFISNITEKNSGLYTCQANNSASGHSRTTVK
TITVSAELPKPSISSNNSKPVEDKDAVAFTCEPEAQNTTYL
WWVNGQSLPVSPRLQLSNGNRTLTLFNVTRNDARAYVCGIQ
NSVSANRSDPVTLDVLYGPDTPIISPPDSSYLSGANLNLSC
HSASNPSPQYSWRINGIPQQHTQVLFIAKITPNNNGTYACF
VSNLATGRNNSIVKSITVSASGTSPGLSAGATVGIMIGVLV
GVALI

Table 7 lists peptide sequences of heavy chain variable domains and light chain variable domains that, in combination, can bind to Claudin-18.2.

TABLE 7

| | Heavy chain | Light chain |
|---|---|---|
| | variable | variable |
| | domain amino | domain amino |
| Clones | acid sequence | acid sequence |
| 1 | QIQLVQSGPELKK | DIVMTQSPSSLT |
| (PCT/EP2015/ | PGETVKISCKAS | VTAGEKVTMSCK |
| 058206) | GYTFTNYGMNWVK | SSQSLLNSGNQK |
| | QAPGKGLKW | NYLTWYQQKPGQ |
| | MGWINTNTGEPTY | PPKLLIYWASTR |
| | AEEFKGRFAFSLE | ESGVPDRFTGSG |
| | TSASTAYLQINN | SGTDFTLTISSV |
| | LKNEDTATYFCAR | QAEDLAVYYCQN |
| | LGFGNAMDYW | DYSYPLTFGAGT |
| | GQGTSVTVSS | KLELK |
| | (SEQ ID | (SEQ ID |
| | NO: 220) | NO: 221) |
| | CDR1 | CDR1 |
| | (SEQ ID | (SEQ ID |
| | NO: 222)- | NO: 225)- |
| | GYTFTNYG | QSLLNSGNQKNY |
| | CDR2 (SEQ ID | CDR2 |
| | NO: 223)- | (SEQ ID |
| | INTNTGEP | NO: 226)- |
| | CDR3 | WAS |
| | (SEQ ID | CDR3 |
| | NO: 224)- | (SEQ ID |
| | ARLGFGNAMDY | NO: 227)- |
| | | QNDYSYPLT |
| 2 | QVQLQQPGAELV | DIVMTQSPSSLT |
| (PCT/EP2015/ | RPGASVKLSCKA | VTAGEKVTMSCK |
| 058206) | SGYTFTSYWINW | SSQSLLNSGNQK |
| | VKQRPGQGLEWI | NYLTWYQQKPGQ |
| | GNIYPSDSYTNY | PPKLLIYWASTR |
| | NQKFKDKATLTV | ESGVPDRFTGSG |
| | DKSSSTAYMQLS | SGTDFTLTISSV |
| | SPTSEDSAVYYC | QAEDLAVYYCQN |
| | TRSWRGNSFDYW | DYSYPFTFGSGT |
| | GQGTTLTVSS | KLEIK |
| | (SEQ ID | (SEQ ID |
| | NO: 228) | NO: 229) |
| | CDR1 | CDR1 |
| | (SEQ ID | (SEQ ID |
| | NO: 230)- | NO: 233)- |
| | GYTFTSYW | QSLLNSGNQKNY |
| | CDR2 | CDR2 |
| | (SEQ ID | (SEQ ID |
| | NO: 231)- | NO: 234)- |
| | IYPSDSYT | WAS |
| | CDR3 | CDR3 |
| | (SEQ ID | (SEQ ID |
| | NO: 232)- | NO: 235)- |
| | TRSWRGNSFDY | QNDYSYPFT |

Alternatively, novel antigen-binding sites that bind to Claudin-18.2 can be identified by screening for binding to the amino acid sequence defined by SEQ ID NO:236.

SEQ ID NO: 236
MAVTACQGLGFVVSLIGIAGIIAATCMDQ
WSTQDLYNNPVTAVFNYQGLWRSCVRESS
GFTECRGYFTLLGLPAMLQAVRALMIVGI
VLGAIGLLVSIFALKCIRIGSMEDSAKAN
MTLTSGIMFIVSGLCAIAGVSVFANMLVT
NFWMSTANMYTGMGGMVQTVQTRYTFGAA
LFVGWVAGGLTLIGGVMMCIACRGLAPEE
TNYKAVSYHASGHSVAYKPGGFKASTGFG
SNTKNKKIYDGGARTEDEVQSYPSKHDYV

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al, Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as an human IgG1 constant region, an IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y4071, Y407V, K409F, K409W, K409D, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

Alternatively, amino acid substitutions could be selected from the following sets of substitutions shown in Table 8.

TABLE 8

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 9.

TABLE 9

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following set of substitutions shown in Table 10.

TABLE 10

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 11.

TABLE 11

| First Polypeptide | Second Polypeptide |
|---|---|
| L351Y, D399R, D399K, | T366V, T366I, T366L, T366M, N390D, |
| S400K, S400R, | N390E, K392L, K392M, K392V, K392F |
| Y407A, Y407I, Y407V | K392D, K392E, K409F, K409W, T411D and |
| | T411E |

Alternatively, at least one amino acid substitutions could be selected from the following set of substitutions in Table 12, where the position(s) indicated in the First Polypeptide column is replaced by any known negatively-charged amino acid, and the position(s) indicated in the Second Polypeptide Column is replaced by any known positively-charged amino acid.

TABLE 12

| First Polypeptide | Second Polypeptide |
|---|---|
| K392, K370, K409, or K439 | D399, E356, or E357 |

Alternatively, at least one amino acid substitutions could be selected from the following set of in Table 13, where the position(s) indicated in the First Polypeptide column is replaced by any known positively-charged amino acid, and the position(s) indicated in the Second Polypeptide Column is replaced by any known negatively-charged amino acid.

TABLE 13

| First Polypeptide | Second Polypeptide |
|---|---|
| D399, E356, or E357 | K409, K439, K370, or K392 |

Alternatively, amino acid substitutions could be selected from the following set in Table 14.

TABLE 14

| First Polypeptide | Second Polypeptide |
|---|---|
| T350V, L351Y, F405A, and Y407V | T350V, T366L, K392L, and T394W |

Alternatively, or in addition, the structural stability of a heteromultimer protein may be increased by introducing S354C on either of the first or second polypeptide chain, and Y349C on the opposing polypeptide chain, which forms an artificial disulfide bridge within the interface of the two polypeptides.

The multi-specific proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; the first, second, and third expression vectors can be stably transfected together into host cells to produce the multimeric proteins.

To achieve the highest yield of the multi-specific protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific protein. The multispecific proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### II. Characteristics of the Multi-Specific Proteins

In certain embodiments, the multi-specific proteins described herein, which include an NKG2D-binding domain and a binding domain for CAIX, ANO1, mesothelin, TROP2, CEA or Claudin-18.2, bind to cells expressing human NKG2D. In certain embodiments, the multi-specific proteins bind to the tumor associated antigen CAIX, ANO1, mesothelin, TROP2, CEA or Claudin-18.2 at a comparable level to that of a monoclonal antibody having the same respective CAIX, ANO1, mesothelin, TROP2, CEA or Claudin-18.2-binding domain. However, the multi-specific proteins described herein may be more effective in reducing tumor growth and killing cancer cells expressing CAIX, ANO1, mesothelin, TROP2, CEA or Claudin-18.2 than the corresponding CAIX, ANO1, mesothelin, TROP2, CEA or Claudin-18.2 monoclonal antibodies.

In certain embodiments, the multi-specific proteins described herein, which include an NKG2D-binding domain and a binding domain for CAIX, ANO1, mesothelin, TROP2, CEA or Claudin-18.2, can activate primary human NK cells when culturing with tumor cells expressing the antigen CAIX, ANO1, mesothelin, TROP2, CEA or Claudin-18.2. NK cell activation is marked by the increase in CD107a degranulation and IFNγ cytokine production.

Furthermore, compared to a monoclonal antibody that includes the same CAIX, ANO1, mesothelin, TROP2, CEA or Claudin-18.2-binding domain, the multi-specific proteins show superior activation of human NK cells in the presence of tumor cells expressing the antigen CAIX, ANO1, mesothelin, TROP2, CEA or Claudin-18.2.

In certain embodiments, the multi-specific proteins described herein, which include an NKG2D-binding domain and a binding domain for CAIX, ANO1, mesothelin, TROP2, CEA or Claudin-18.2, can enhance the activity of rested and IL-2-activated human NK cells in the presence of tumor cells expressing the antigen CAIX, ANO1, mesothelin, TROP2, CEA or Claudin-18.2.

In certain embodiments, the multi-specific proteins described herein, which include an NKG2D-binding domain and a binding domain for tumor-associated antigen CAIX, ANO1, mesothelin, TROP2, CEA or Claudin-18.2, can enhance the cytotoxic activity of rested and IL-2-activated human NK cells in the presence of tumor cells expressing the antigen CAIX, ANO1, mesothelin, TROP2, CEA or Claudin-18.2. In certain embodiments, compared to the corresponding monoclonal antibodies, the multi-specific proteins can offer an advantage against tumor cells expressing medium and low CAIX, ANO1, mesothelin, TROP2, CEA or Claudin-18.2.

In certain embodiments, the multi-specific proteins described herein can be advantageous in treating cancers with high expression of Fc receptor (FcR), or cancers residing in a tumor microenvironment with high levels of FcR, compared to the corresponding CAIX, ANO1, mesothelin, TROP2, CEA or Claudin-18.2 monoclonal antibodies. Monoclonal antibodies exert their effects on tumor growth through multiple mechanisms including ADCC, CDC, phagocytosis, and signal blockade amongst others. Amongst FcγRs, CD16 has the lowest affinity for IgG Fc; FcγRI (CD64) is the high-affinity FcR, which binds about 1000 times more strongly to IgG Fc than CD16. CD64 is normally expressed on many hematopoietic lineages such as the myeloid lineage, and can be expressed on tumors derived from these cell types, such as acute myeloid leukemia (AML) Immune cells infiltrating into the tumor, such as MDSCs and monocytes, also express CD64 and are known to infiltrate the tumor microenvironment. Expression of CD64 by the tumor or in the tumor microenvironment can have a detrimental effect on monoclonal antibody therapy. Expression of CD64 in the tumor microenvironment makes it difficult for these antibodies to engage CD16 on the surface of NK cells, as the antibodies prefer to bind the high-affinity receptor. The multi-specific proteins, through targeting two activating receptors on the surface of NK cells, can overcome the detrimental effect of CD64 expression (either on tumor or tumor microenvironment) on monoclonal antibody therapy. Regardless of CD64 expression on the tumor cells, the multi-specific proteins are able to mediate human NK cell responses against all tumor cells, because dual targeting of two activating receptors on NK cells provides stronger specific binding to NK cells.

In some embodiments, the multi-specific proteins described herein can provide a better safety profile through reduced on-target off-tumor side effects. Natural killer cells and CD8 T cells are both able to directly lyse tumor cells, although the mechanisms through which NK cells and CD8 T cells recognize normal self from tumor cells differ. The activity of NK cells is regulated by the balance of signals from activating (NCRs, NKG2D, CD16, etc.) and inhibitory (KIRs, NKG2A, etc.) receptors. The balance of these activating and inhibitory signals allow NK cells to determine healthy self-cells from stressed, virally infected, or transformed self-cells. This “built-in” mechanism of self-tolerance will help protect normal heathy tissue from NK cell responses. To extend this principle, the self-tolerance of NK cells will allow TriNKETs to target antigens expressed both on self and tumor without off-tumor side effects, or with an increased therapeutic window. Unlike natural killer cells, T cells require recognition of a specific peptide presented by MHC molecules for activation and effector functions. T cells have been the primary target of immunotherapy, and many strategies have been developed to redirect T cell responses against the tumor. T cell bispecifics, checkpoint inhibitors, and CAR-T cells have all been approved by the FDA, but often suffer from dose-limiting toxicities. T cell bispecifics and CAR-T cells work around the TCR-MHC recognition system by using binding domains to target antigens on the surface of tumor cells, and using engineered signaling domains to transduce the activation signals into the effector cell. Although effective at eliciting an anti-tumor immune response these therapies are often coupled with cytokine release syndrome (CRS), and on-target off-tumor side effects. The multi-specific proteins are unique in this context as they will not “override” the natural systems of NK cell activation and inhibition. Instead, the multi-specific proteins are designed to sway the balance, and provide additional activation signals to the NK cells, while maintaining NK tolerance to healthy self.

In some embodiments, the multi-specific proteins described herein can delay progression of the tumor more effectively than the corresponding CAIX, ANO1, mesothelin, TROP2, CEA or Claudin-18.2 monoclonal antibodies that include the same antigen-binding domain. In some embodiments, the multi-specific proteins described herein can be more effective against cancer metastases than the corresponding CAIX, ANO1, mesothelin, TROP2, CEA or Claudin-18.2 monoclonal antibodies that include the same antigen-binding domain.

### III. Therapeutic Applications

The invention provides methods for treating cancer using a multi-specific binding protein described herein and/or a pharmaceutical composition described herein. The methods may be used to treat a variety of cancers which express CAIX, ANO1, mesothelin, TROP2, CEA or Claudin-18.2 by administering to a patient in need thereof a therapeutically effective amount of a multi-specific binding protein described herein.

The therapeutic method can be characterized according to the cancer to be treated. Cancers to be treated using CAIX-targeting multi-specific binding proteins include, for example, renal cell carcinoma, breast cancer, glioblastoma, head and neck cancer, gastric cancers, bladder cancer, ovarian cancer, tumors of the esophagus, lung, colon, kidney, cervix and non-small cell lung carcinoma. Cancers to be treated using mesothelin-targeting multi-specific binding proteins include, for example, mesothelioma, ovarian cancer, pancreatic cancer, non-small cell lung cancer. breast cancer, cholangiocarcinoma, gastric cancer, uterine serous carcinoma, thymic carcinoma, and acute myeloid leukemia. Cancers to be treated using ANO1-targeting multi-specific binding proteins include, for example, esophageal squamous cell cancer (ESCC), gastrointestinal stromal tumor (GIST), head and neck squamous cell carcinoma (HNSCC), pancreatic cancer, breast cancer, prostate cancer, and sarcoma. Cancers to be treated using TROP2-targeting multi-specific binding proteins include, for example, breast, lung, gastric, colorectal, pancreatic, prostatic, cervical, head-and-neck cancer, nasopharyngeal carcinoma, and ovarian carcinoma. Cancers to be treated using CEA-targeting multi-specific binding proteins include, for example, gastrointestinal cancer, colorectal cancer, pancreatic cancer, non-small cell lung cancer and breast cancer. Cancers to be treated using Claudin-18.2-targeting multi-specific binding proteins include, for example, esophageal cancer, non-small cell lung carcinoma, ovarian cancer, colon cancer, and several forms of biliary ductal carcinoma.

In certain other embodiments, the cancer is brain cancer, cervical cancer, colon cancer, colorectal cancer, endometrial cancer, esophageal cancer, leukemia, lung cancer, liver cancer, melanoma, ovarian cancer, pancreatic cancer, rectal cancer, renal cancer, stomach cancer, testicular cancer, or uterine cancer. In yet other embodiments, the cancer is a squamous cell carcinoma, adenocarcinoma, small cell carcinoma, melanoma, neuroblastoma, sarcoma (e.g., an angiosarcoma or chondrosarcoma), larynx cancer, parotid cancer, bilary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The cancer to be treated can be characterized according to the presence of a particular antigen expressed on the surface of the cancer cell. In certain embodiments, the cancer cell can express one or more of the following: CD2, CD19, CD20, CD30, CD38, CD40, CD52, CD70, EGFR/ERBB1, IGF1R, HER3/ERBB3, HER4/ERBB4, MUC1, cMET, SLAMF7, PSCA, MICA, MICB, TRAILR1, TRAILR2, MAGE-A3, B7.1, B7.2, CTLA4, and PD1.

### IV. Combination Therapy

Another aspect of the invention provides for combination therapy. Multi-specific binding proteins described herein be used in combination with additional therapeutic agents to treat the cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAGS, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDCl₇ Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the invention can also be used as an adjunct to surgical removal of the primary lesion.

The amount of multi-specific binding protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

### V. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The intravenous drug delivery formulation of the present disclosure may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may be freeze-dried (lyophilized) and contained in about 12-60 vials. In certain embodiments, the formulation may be freeze-dried and 45 mg of the freeze-dried formulation may be contained in one vial. In certain embodiments, the about 40 mg-about 100 mg of freeze-dried formulation may be contained in one vial. In certain embodiments, freeze-dried formulations from 12, 27, or 45 vials are combined to obtained a therapeutic dose of the protein in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial.

This present disclosure could exist in a liquid aqueous pharmaceutical formulation including a therapeutically effective amount of the protein in a buffered solution forming a formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/ml of citric acid (e.g., 1.305 mg/ml), about 0.3 mg/ml of sodium citrate (e.g., 0.305 mg/ml), about 1.5 mg/ml of disodium phosphate dihydrate (e.g., 1.53 mg/ml), about 0.9 mg/ml of sodium dihydrogen phosphate dihydrate (e.g., 0.86), and about 6.2 mg/ml of sodium chloride (e.g., 6.165 mg/ml). In certain embodiments, the buffer system includes 1-1.5 mg/ml of citric acid, 0.25 to 0.5 mg/ml of sodium citrate, 1.25 to 1.75 mg/ml of disodium phosphate dihydrate, 0.7 to 1.1 mg/ml of sodium dihydrogen phosphate dihydrate, and 6.0 to 6.4 mg/ml of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/ml. In certain embodiments, the concentration of mannitol may be about 7.5 to 15 mg/ml. In certain embodiments, the concentration of mannitol may be about 10-14 mg/ml. In certain embodiments, the concentration of mannitol may be about 12 mg/ml. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, the protein product of the present disclosure is formulated as a liquid formulation. The liquid formulation may be presented at a 10 mg/mL concentration in either a USP/Ph Eur type I 5OR vial closed with a rubber stopper and sealed with an aluminum crimp seal closure. The stopper may be made of elastomer complying with USP and Ph Eur. In certain embodiments vials may be filled with 61.2 mL of the protein product solution in order to allow an extractable volume of 60 mL. In certain embodiments, the liquid formulation may be diluted with 0.9% saline solution.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation of the present disclosure may be preserved under conditions of pH and humidity to prevent deamination of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A preservative may be optionally added to the formulations herein to reduce bacterial action. The addition of a preservative may, for example, facilitate the production of a multi-use (multiple-dose) formulation.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

This present disclosure could exist in a lyophilized formulation including the proteins and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between 6 to 8. In certain embodiments, the pH range for the lyophilized drug product may be from 7 to 8.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution.

In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product of the instant disclosure is constituted to about 4.5 mL water for injection and diluted with 0.9% saline solution (sodium chloride solution).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of the invention. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and is not intended to limit the invention.

### Example 1—NKG2D Binding Domains Bind to NKG2D

### NKG2D Binding Domains Bind to Purified Recombinant NKG2D

The nucleic acid sequences of human, mouse or cynomolgus NKG2D ectodomains were fused with nucleic acid sequences encoding human IgG1 Fc domains and introduced into mammalian cells to be expressed. After purification, NKG2D-Fc fusion proteins were adsorbed to wells of microplates. After blocking the wells with bovine serum albumin to prevent non-specific binding, NKG2D-binding domains were titrated and added to the wells pre-adsorbed with NKG2D-Fc fusion proteins. Primary antibody binding was detected using a secondary antibody which was conjugated to horseradish peroxidase and specifically recognizes a human kappa light chain to avoid Fc cross-reactivity. 3,3′,5,5′-Tetramethylbenzidine (TMB), a substrate for horseradish peroxidase, was added to the wells to visualize the binding signal, whose absorbance was measured at 450 nM and corrected at 540 nM. An NKG2D-binding domain clone, an isotype control or a positive control (selected from SEQ ID NOs:47-50, or anti-mouse NKG2D clones MI-6 and CX-5 available at eBioscience) was added to each well.

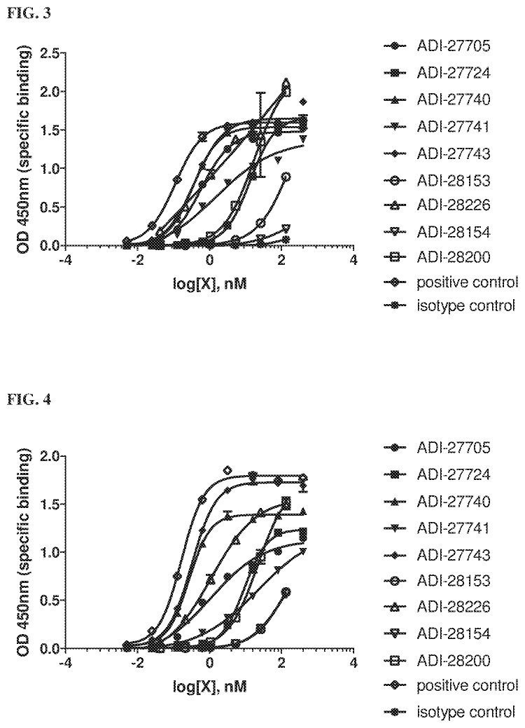

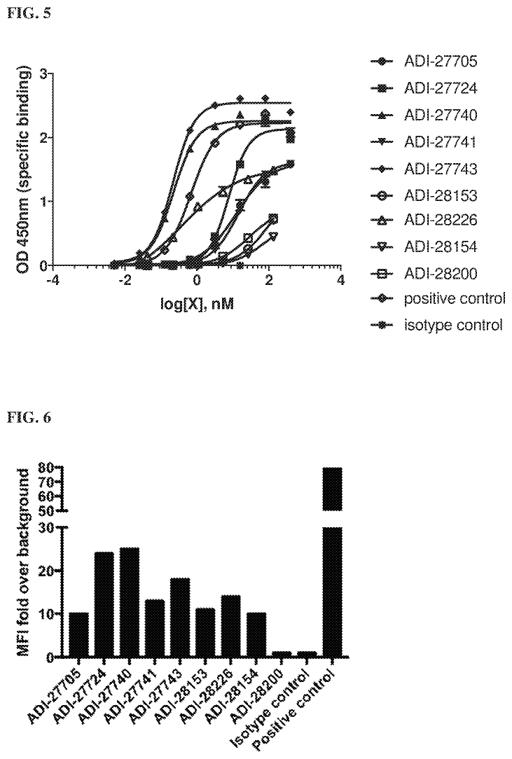

The isotype control showed minimal binding to recombinant NKG2D-Fc proteins, while the positive control bound strongest to the recombinant antigens. NKG2D-binding domains produced by all clones demonstrated binding across human, mouse, and cynomolgus recombinant NKG2D-Fc proteins, although with varying affinities from clone to clone. Generally, each anti-NKG2D clone bound to human (FIG. 3) and cynomolgus (FIG. 4) recombinant NKG2D-Fc with similar affinity, but with lower affinity to mouse (FIG. 5) recombinant NKG2D-Fc.

### NKG2D-Binding Domains Bind to Cells Expressing NKG2D

EL4 mouse lymphoma cell lines were engineered to express human or mouse NKG2D-CD3 zeta signaling domain chimeric antigen receptors. An NKG2D-binding clone, an isotype control or a positive control was used at a 100 nM concentration to stain extracellular NKG2D expressed on the EL4 cells. The antibody binding was detected using fluorophore-conjugated anti-human IgG secondary antibodies. Cells were analyzed by flow cytometry, and fold-over-background (FOB) was calculated using the mean fluorescence intensity (MFI) of NKG2D expressing cells compared to parental EL4 cells.

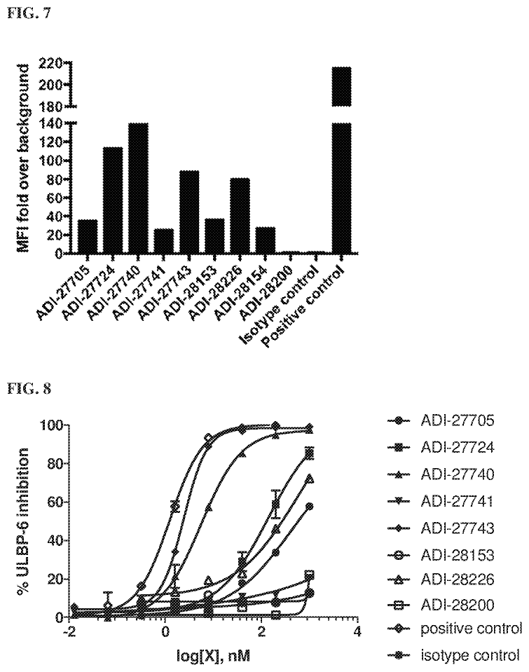

NKG2D-binding domains produced by all clones bound to EL4 cells expressing human and mouse NKG2D. Positive control antibodies (selected from SEQ ID NO: 45-48, or anti-mouse NKG2D clones MI-6 and CX-5 available at eBioscience) gave the best FOB binding signal. The NKG2D-binding affinity for each clone was similar between cells expressing human NKG2D (FIG. 6) and mouse (FIG. 7) NKG2D.

### Example 2—NKG2D-Binding Domains Block Natural Ligand Binding to NKG2D

### Competition With ULBP-6

Recombinant human NKG2D-Fc proteins were adsorbed to wells of a microplate, and the wells were blocked with bovine serum albumin reduce non-specific binding. A saturating concentration of ULBP-6-His-biotin was added to the wells, followed by addition of the NKG2D-binding domain clones. After a 2-hour incubation, wells were washed and ULBP-6-His-biotin that remained bound to the NKG2D-Fc coated wells was detected by streptavidin-conjugated to horseradish peroxidase and TMB substrate. Absorbance was measured at 450 nM and corrected at 540 nM. After subtracting background, specific binding of NKG2D-binding domains to the NKG2D-Fc proteins was calculated from the percentage of ULBP-6-His-biotin that was blocked from binding to the NKG2D-Fc proteins in wells. The positive control antibody (selected from SEQ ID NOs:47-50) and various NKG2D-binding domains blocked ULBP-6 binding to NKG2D, while isotype control showed little competition with ULBP-6 (FIG. 8).

ULBP-6 sequence is represented by SEQ ID NO:131

(SEQ ID NO: 131)
MAAAAIPALLLCLPLLFLLFGWSRARRDD
PHSLCYDITVIPKFRPGPRWCAVQGQVDE
KTFLHYDCGNKTVTPVSPLGKKLNVTMAW
KAQNPVLREVVDILTEQLLDIQLENYTPK
EPLTLQARMSCEQKAEGHSSGSWQFSIDG
QTFLLFDSEKRMWTTVHPGARKMKEKWEN
DKDVAMSFHYISMGDCIGWLEDFLMGMDS
TLEPSAGAPLAMSSGTTQLRATATTLILC
CLLIILPCFILPGI

Competition with MICA

Recombinant human MICA-Fc proteins were adsorbed to wells of a microplate, and the wells were blocked with bovine serum albumin to reduce non-specific binding. NKG2D-Fc-biotin was added to wells followed by NKG2D-binding domains. After incubation and washing, NKG2D-Fc-biotin that remained bound to MICA-Fc coated wells was detected using streptavidin-HRP and TMB substrate. Absorbance was measured at 450 nM and corrected at 540 nM. After subtracting background, specific binding of NKG2D-binding domains to the NKG2D-Fc proteins was calculated from the percentage of NKG2D-Fc-biotin that was blocked from binding to the MICA-Fc coated wells. The positive control antibody (selected from SEQ ID NOs:47-50) and various NKG2D-binding domains blocked MICA binding to NKG2D, while isotype control showed little competition with MICA (FIG. 9).

Competition with Rae-1 Delta

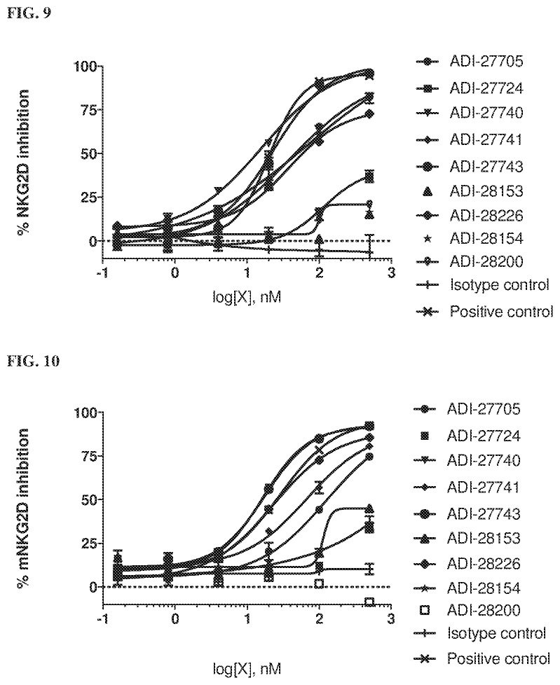

Recombinant mouse Rae-1 delta-Fc (purchased from R&D Systems) was adsorbed to wells of a microplate, and the wells were blocked with bovine serum albumin to reduce non-specific binding. Mouse NKG2D-Fc-biotin was added to the wells followed by NKG2D-binding domains. After incubation and washing, NKG2D-Fc-biotin that remained bound to Rae-1delta-Fc coated wells was detected using streptavidin-HRP and TMB substrate. Absorbance was measured at 450 nM and corrected at 540 nM. After subtracting background, specific binding of NKG2D-binding domains to the NKG2D-Fc proteins was calculated from the percentage of NKG2D-Fc-biotin that was blocked from binding to the Rae-1delta-Fc coated wells. The positive control (selected from SEQ ID NOs:47-50, or anti-mouse NKG2D clones MI-6 and CX-5 available at eBioscience) and various NKG2D-binding domain clones blocked Rae-1delta binding to mouse NKG2D, while the isotype control antibody showed little competition with Rae-1delta (FIG. 10).

### Example 3—NKG2D-Binding Domain Clones Activate NKG2D

Nucleic acid sequences of human and mouse NKG2D were fused to nucleic acid sequences encoding a CD3 zeta signaling domain to obtain chimeric antigen receptor (CAR) constructs. The NKG2D-CAR constructs were then cloned into a retrovirus vector using Gibson assembly and transfected into expi293 cells for retrovirus production. EL4 cells were infected with viruses containing NKG2D-CAR together with 8 μg/mL polybrene. 24 hours after infection, the expression levels of NKG2D-CAR in the EL4 cells were analyzed by flow cytometry, and clones which express high levels of the NKG2D-CAR on the cell surface were selected.

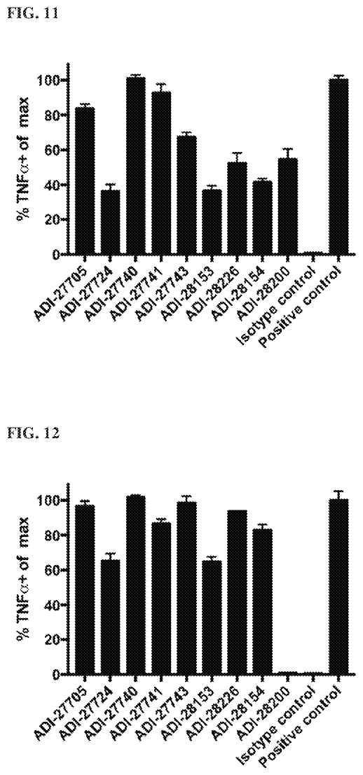

To determine whether NKG2D-binding domains activate NKG2D, they were adsorbed to wells of a microplate, and NKG2D-CAR EL4 cells were cultured on the antibody fragment-coated wells for 4 hours in the presence of brefeldin-A and monensin. Intracellular TNF-alpha production, an indicator for NKG2D activation, was assayed by flow cytometry. The percentage of TNF-alpha positive cells was normalized to the cells treated with the positive control. All NKG2D-binding domains activated both human NKG2D (FIG. 11) and mouse NKG2D (FIG. 12).

### Example 4—NKG2D-Binding Domains Activate NK Cells

### Primary Human NK Cells

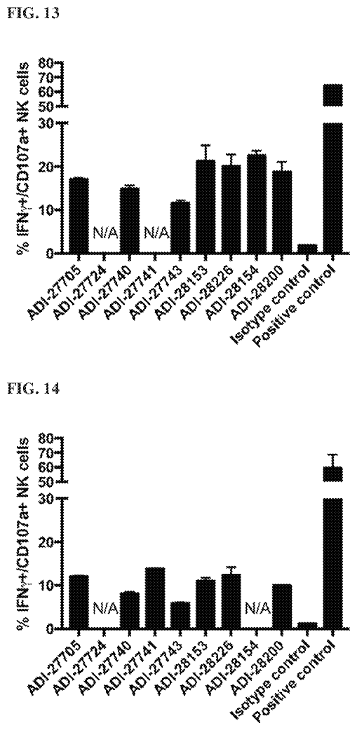

Peripheral blood mononuclear cells (PBMCs) were isolated from human peripheral blood buffy coats using density gradient centrifugation. NK cells (CD3⁻CD56⁺) were isolated using negative selection with magnetic beads from PBMCs, and the purity of the isolated NK cells was typically >95%. Isolated NK cells were then cultured in media containing 100 ng/mL IL-2 for 24-48 hours before they were transferred to the wells of a microplate to which the NKG2D-binding domains were adsorbed, and cultured in the media containing fluorophore-conjugated anti-CD107a antibody, brefeldin-A, and monensin. Following culture, NK cells were assayed by flow cytometry using fluorophore-conjugated antibodies against CD3, CD56 and IFN-γ. CD107a and IFN-γ staining were analyzed in CD3⁻CD56⁺ cells to assess NK cell activation. The increase in CD107a/IFN-γ double-positive cells is indicative of better NK cell activation through engagement of two activating receptors rather than one receptor. NKG2D-binding domains and the positive control (selected from SEQ ID NOs:47-50) showed a higher percentage of NK cells becoming CD107a⁺ and IFN-γ⁺ than the isotype control (FIG. 13 & FIG. 14 represent data from two independent experiments, each using a different donor's PBMC for NK cell preparation).

### Primary Mouse NK Cells

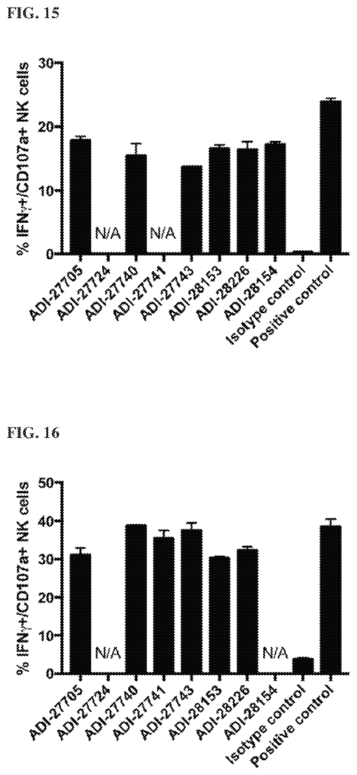

Spleens were obtained from C57Bl/6 mice and crushed through a 70 μm cell strainer to obtain single cell suspension. Cells were pelleted and resuspended in ACK lysis buffer (purchased from Thermo Fisher Scientific #A1049201; 155 mM ammonium chloride, 10 mM potassium bicarbonate, 0.01 mM EDTA) to remove red blood cells. The remaining cells were cultured with 100 ng/mL hIL-2 for 72 hours before being harvested and prepared for NK cell isolation. NK cells (CD3⁻NK1.1⁺) were then isolated from spleen cells using a negative depletion technique with magnetic beads with typically >90% purity. Purified NK cells were cultured in media containing 100 ng/mL mIL-15 for 48 hours before they were transferred to the wells of a microplate to which the NKG2D-binding domains were adsorbed, and cultured in the media containing fluorophore-conjugated anti-CD107a antibody, brefeldin-A, and monensin. Following culture in NKG2D-binding domain-coated wells, NK cells were assayed by flow cytometry using fluorophore-conjugated antibodies against CD3, NK1.1 and IFN-γ. CD107a and IFN-γ staining were analyzed in CD3⁻NK1.1⁺ cells to assess NK cell activation. The increase in CD107a/IFN-γ double-positive cells is indicative of better NK cell activation through engagement of two activating receptors rather than one receptor. NKG2D-binding domains and the positive control (selected from anti-mouse NKG2D clones MI-6 and CX-5 available at eBioscience) showed a higher percentage of NK cells becoming CD107a⁺ and IFN-γ⁺ than the isotype control (FIG. 15 & FIG. 16 represent data from two independent experiments, each using a different mouse for NK cell preparation).

### Example 5—NKG2D-Binding Domains Enable Cytotoxicity of Target Tumor Cells

Human and mouse primary NK cell activation assays demonstrate increased cytotoxicity markers on NK cells after incubation with NKG2D-binding domains. To address whether this translates into increased tumor cell lysis, a cell-based assay was utilized where each NKG2D-binding domain was developed into a monospecific antibody. The Fc region was used as one targeting arm, while the Fab region (NKG2D-binding domain) acted as another targeting arm to activate NK cells. THP-1 cells, which are of human origin and express high levels of Fc receptors, were used as a tumor target and a Perkin Elmer DELFIA Cytotoxicity Kit was used. THP-1 cells were labeled with BATDA reagent, and resuspended at 10⁵/mL in culture media. Labeled THP-1 cells were then combined with NKG2D antibodies and isolated mouse NK cells in wells of a microtiter plate at 37° C. for 3 hours. After incubation, 20 μl of the culture supernatant was removed, mixed with 200 μl of Europium solution and incubated with shaking for 15 minutes in the dark. Fluorescence was measured over time by a PheraStar plate reader equipped with a time-resolved fluorescence module (Excitation 337 nm, Emission 620 nm) and specific lysis was calculated according to the kit instructions.

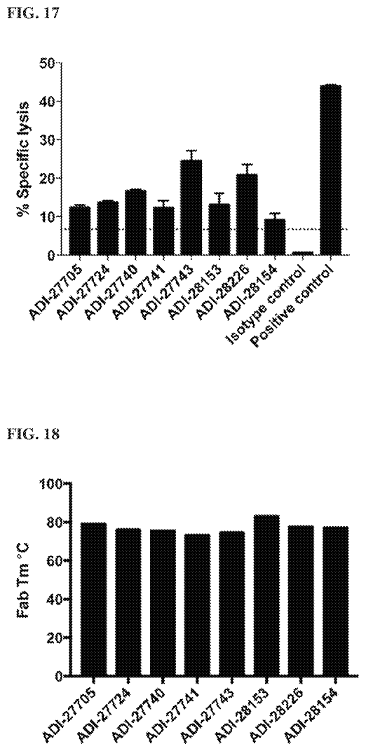

The positive control, ULBP-6—a natural ligand for NKG2D, showed increased specific lysis of THP-1 target cells by mouse NK cells. NKG2D antibodies also increased specific lysis of THP-1 target cells, while isotype control antibody showed reduced specific lysis. The dotted line indicates specific lysis of THP-1 cells by mouse NK cells without antibody added (FIG. 17).

### Example 6—NKG2D Antibodies Show High Thermostability

Melting temperatures of NKG2D-binding domains were assayed using differential scanning fluorimetry. The extrapolated apparent melting temperatures are high relative to typical IgG1 antibodies (FIG. 18).

### Example 7—Synergistic Activation of Human NK Cells by Cross-Linking NKG2D and CD16

### Primary Human NK Cell Activation Assay

Peripheral blood mononuclear cells (PBMCs) were isolated from peripheral human blood buffy coats using density gradient centrifugation. NK cells were purified from PBMCs using negative magnetic beads (StemCell #17955). NK cells were >90% CD3⁻CD56⁺ as determined by flow cytometry. Cells were then expanded 48 hours in media containing 100 ng/mL hIL-2 (Peprotech #200-02) before use in activation assays. Antibodies were coated onto a 96-well flat-bottom plate at a concentration of 2 μg/ml (anti-CD16, Biolegend #302013) and 5 μg/mL (anti-NKG2D, R&D #MAB139) in 100 μl sterile PBS overnight at 4° C. followed by washing the wells thoroughly to remove excess antibody. For the assessment of degranulation IL-2-activated NK cells were resuspended at 5×10⁵ cells/ml in culture media supplemented with 100 ng/mL hIL2 and 1 μg/mL APC-conjugated anti-CD107a mAb (Biolegend #328619). 1×10⁵ cells/well were then added onto antibody coated plates. The protein transport inhibitors Brefeldin A (BFA, Biolegend #420601) and Monensin (Biolegend #420701) were added at a final dilution of 1:1000 and 1:270 respectively. Plated cells were incubated for 4 hours at 37° C. in 5% CO₂. For intracellular staining of IFN-γ NK cells were labeled with anti-CD3 (Biolegend #300452) and anti-CD56 mAb (Biolegend #318328) and subsequently fixed and permeabilized and labeled with anti-IFN-γ mAb (Biolegend #506507). NK cells were analyzed for expression of CD107a and IFN-γ by flow cytometry after gating on live CD56⁺CD3⁻ cells.

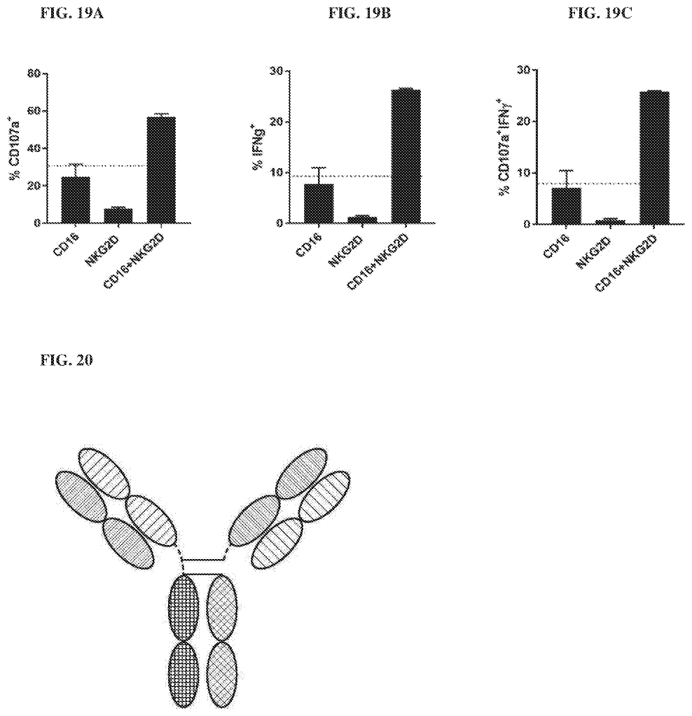

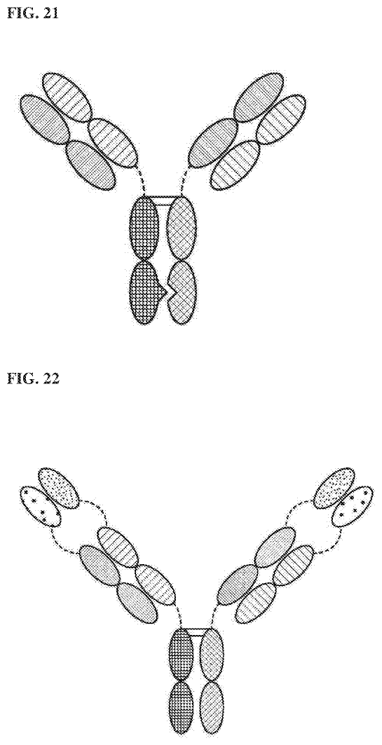

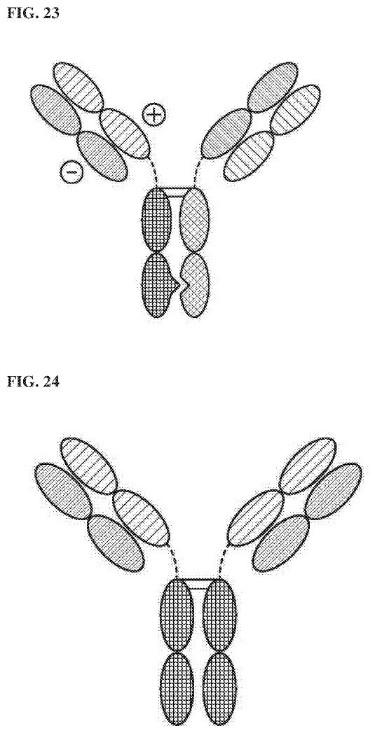

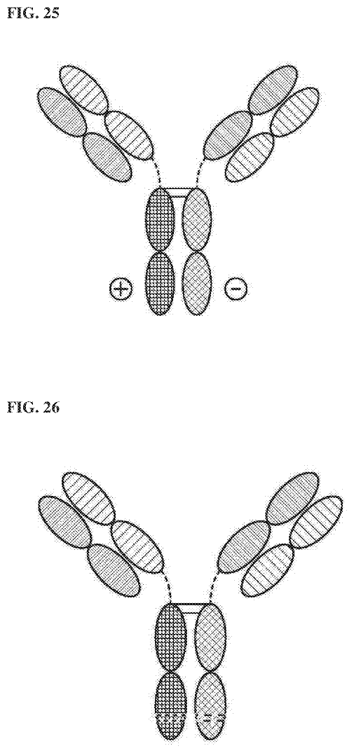

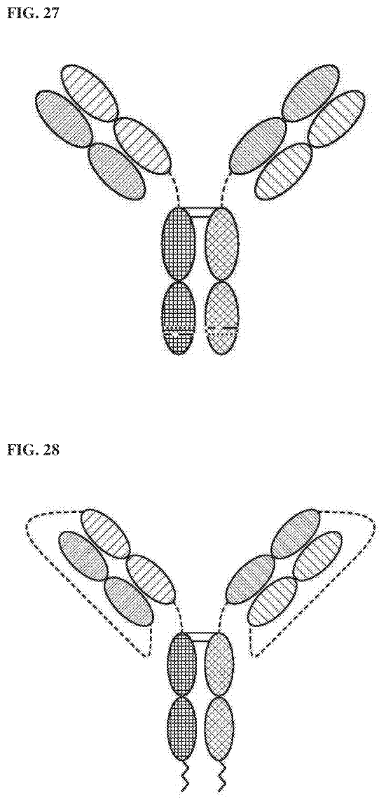

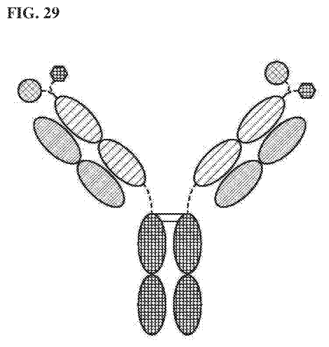

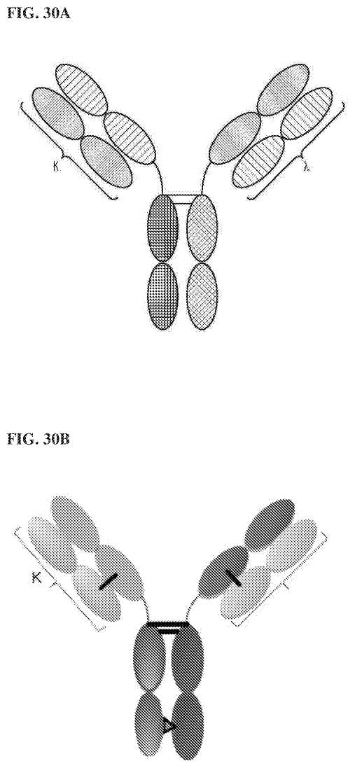

To investigate the relative potency of receptor combination, crosslinking of NKG2D or CD16 and co-crosslinking of both receptors by plate-bound stimulation was performed. As shown in FIG. 19 (FIGS. 19A-19C), combined stimulation of CD16 and NKG2D resulted in highly elevated levels of CD107a (degranulation) (FIG. 19A) and/or IFN-γ production (FIG. 19B). Dotted lines represent an additive effect of individual stimulations of each receptor.

CD107a levels and intracellular IFN-γ production of IL-2-activated NK cells were analyzed after 4 hours of plate-bound stimulation with anti-CD16, anti-NKG2D or a combination of both monoclonal antibodies. Graphs indicate the mean (n=2)±SD. FIG. 19A demonstrates levels of CD107a; FIG. 19B demonstrates levels of IFNγ; FIG. 19C demonstrates levels of CD107a and IFNγ. Data shown in FIGS. 19A-19C are representative of five independent experiments using five different healthy donors.

### Example 8—Multi-Specific Binding Proteins Bind to NKG2D

EL4 mouse lymphoma cell lines were engineered to express human NKG2D. Trispecific binding proteins (TriNKETs) that each contain an NKG2D-binding domain, a tumor-associated antigen-binding domain (CAIX-binding domain), and an Fc domain that binds to CD16 as shown in FIG. 1, were tested for their affinity to extracellular NKG2D expressed on EL4 cells. The binding of the multi-specific binding proteins to NKG2D was detected using fluorophore-conjugated anti-human IgG secondary antibodies. Cells were analyzed by flow cytometry, and fold-over-background (FOB) was calculated using the mean fluorescence intensity (MFI) of NKG2D-expressing cells compared to parental EL4 cells.

TriNKETs tested include A44-TriNKET-CAIX (ADI-27744 and a CAIX-binding domain), F63-TriNKET-CAIX (ADI-29404 and a CD33-binding domain), E79-TriNKET-CAIX (ADI-29379 and a CAIX-binding domain), F47-TriNKET-CAIX (ADI-29447 and a CAIX-binding domain), and A49-TriNKET-CAIX (ADI-27749 and a CAIX-binding domain). The CAIX-binding domain was composed of a heavy chain variable domain of SEQ ID NO:80, and light chain variable domain of SEQ ID NO:81.

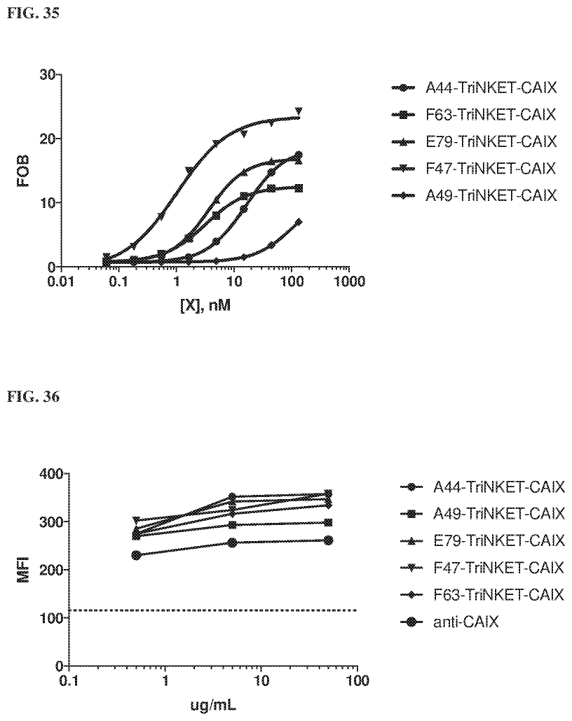

FIG. 35 shows that TriNKETs, each of which includes a CAIX-binding domain and a distinct NKG2D-binding domain, bind to NKG2D expressed on EL4 cells.

### Example 9—Multi-Specific Binding Proteins Bind to Human Tumor Antigens

Trispecific binding proteins bind to CAIX

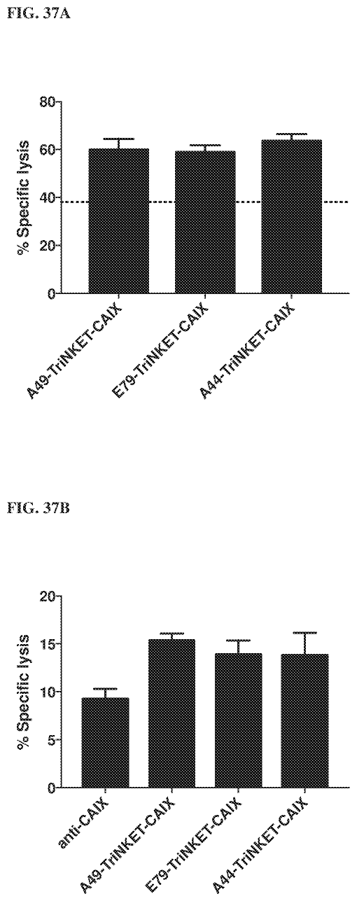

Human renal cell carcinoma line A498 expressing CAIX was used to assay the binding of TriNKETs to the tumor associated antigen CAIX. TriNKETs and optionally the parental anti-CAIX monoclonal antibody were incubated with the cells, and the binding was detected using fluorophore-conjugated anti-human IgG secondary antibodies. Cells were analyzed by flow cytometry, and fold-over-background (FOB) was calculated using the mean fluorescence intensity (MFI) from the TriNKETs and the parental monoclonal anti-CAIX antibody normalized to secondary antibody controls. CAIX-targeting TriNKETs show comparable levels of binding to CAIX on A498 cells as compared with the parental anti-CAIX antibody (FIG. 36). The overall binding signal is comparable among TriNKETs as they contain the same CAIX binding domain.

### Example 10—Trispecific Binding Proteins Enable Cytotoxicity of Target Cancer Cells

Peripheral blood mononuclear cells (PBMCs) were isolated from human peripheral blood buffy coats using density gradient centrifugation. NK cells (CD3⁻CD56⁺) were isolated using negative selection with magnetic beads from PBMCs, and the purity of the isolated NK cells was typically >90%. Isolated NK cells were cultured in media containing 100 ng/mL IL-2 for activation or rested overnight without cytokine. IL-2-activated or rested NK cells were used the following day in cytotoxicity assays.

NK/NK T-cell lines KHYG-1 cells were maintained in 10% HI-FBS-RPMI-1640 with 10 ng/mL IL-2. The day before use as effector cells, KHYG-1 cells were harvested from culture, and cells were washed out of the IL-2 containing media. After washing KHYG-1 cells were resuspended in 10% HI-FBS-RPMI-1640, and were rested overnight without cytokine.

### DELFIA Cytotoxicity Assay:

Human A498 cancer cells expressing CAIX were harvested from culture; cells were washed with PBS and were resuspended in growth media at 10⁶/mL for labeling with BATDA reagent (Perkin Elmer AD0116). Manufacturer instructions were followed for labeling of the target cells. After labeling cells were washed 3× with PBS, and were resuspended at 0.5-1.0×10⁵/mL in culture media. To prepare the background wells an aliquot of the labeled cells was put aside, and the cells were spun out of the media. 100 μl of the media was carefully added to wells in triplicate to avoid disturbing the pelleted cells. 100 μl of BATDA-labeled cells were added to each well of the 96-well plate. Wells were saved for spontaneous release from target cells, and wells were prepared for max lysis of target cells by addition of 1% Triton-X. TriNKETs against the tumor target of interest were diluted in culture media, 50 μl of diluted TriNKET was added to each well. Rested NK cells, activated NK cells or KHYG-1 cells were harvested from culture, washed, and resuspended at 10⁵-2.0×10⁶/mL in culture media depending on the desired effector to target cell ratio (E:T). 50 μl of NK cells was added to each well of the plate to make a total of 200 μl culture volume. The plate was incubated at 37° C. with 5% CO₂ for 2-3 hours before developing the assay.

After culturing for 2-3 hours, the plate was removed from the incubator and the cells were pelleted by centrifugation at 200 g for 5 minutes. 20 μl of culture supernatant was transferred to a clean microplate provided from the manufacturer and 200 μl of room temperature europium solution was added to each well. The plate was protected from the light and incubated on a plate shaker at 250 rpm for 15 minutes. The plate was read using either Victor 3 or SpectraMax i3X instruments. % Specific lysis was calculated as follows: % Specific lysis=((Experimental release−Spontaneous release)/(Maximum release−Spontaneous release))*100%.

TriNKETs mediated cytotoxicity of human NK cells towards the CAIX-positive human cancer cells. Co-culture of rested KHYG-1 cells with A498 target cells resulted in −38% specific lysis tumor lysis (shown by the dotted line in FIG. 37A). Addition of three different TriNKETs targeting CAIX (A49-TriNKET-CAIX; E79-TriNKET-CALX; and A44-TriNKET-CAIX) to the co-culture enhanced specific lysis of tumor cells by about 20% for all three NKG2D binding domains (FIG. 37A).

Rested human NK cells were mixed with A498 cancer cells. Rested human NK cells had no cytotoxic activity against A498 cells without CAIX mAb or TriNKETs added. A monoclonal antibody against CAIX (BAY79-4620 variable domains with hIgG1 as mAb (homodimer) was able to increase specific lysis to about 9%. TriNKETs (e.g., A49-TriNKET-CAIX, E79-TriNKET-CAIX and A44-TriNKET-CAIX) enhanced cytotoxic activity of rested human NK cells towards the cancer cells compared to the monoclonal antibody (FIG. 37B).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A protein comprising:
(a) a first antigen-binding site that binds NKG2D;
(b) a second antigen-binding site that binds CEA; and
(c) an antibody Fc domain or a portion thereof sufficient to bind CD16, or a third antigen-binding site that binds CD16.

2. The protein of claim 1, wherein the first antigen-binding site binds to NKG2D in humans and non-human primates.

3. The protein of claim 1 or 2, wherein the first antigen-binding site comprises a heavy chain variable domain and a light chain variable domain.

4. The protein according to claim 3, wherein the heavy chain variable domain and the light chain variable domain are present on the same polypeptide.

5. The protein according to claim 3 or 4, wherein the second antigen-binding site comprises a heavy chain variable domain and a light chain variable domain.

6. The protein according to claim 5, wherein the heavy chain variable domain and the light chain variable domain of the second antigen-binding site are present on the same polypeptide.

7. The protein according to claim 5 or 6, wherein the light chain variable domain of the first antigen-binding site has an amino acid sequence identical to the amino acid sequence of the light chain variable domain of the second antigen-binding site.

8. A protein according to any one of the preceding claims, wherein the first antigen-binding site comprises a heavy chain variable domain amino acid sequence at least 90% identical to SEQ ID NO:1.

9. The protein according to any one of claims 1-7, wherein the first antigen-binding site comprises a heavy chain variable domain amino acid sequence at least 90% identical to SEQ ID NO:41 and a light chain variable domain amino acid sequence at least 90% identical to SEQ ID NO:42.

10. The protein according to any one of claims 1-7, wherein the first antigen-binding site comprises a heavy chain variable domain amino acid sequence at least 90% identical to SEQ ID NO:43 and a light chain variable domain amino acid sequence at least 90% identical to SEQ ID NO:44.

11. The protein according to any one of claims 1-7, wherein the first antigen-binding site comprises a heavy chain variable domain amino acid sequence at least 90% identical to SEQ ID NO:45 and a light chain variable domain amino acid sequence at least 90% identical to SEQ ID NO:46.

12. The protein according to any one of claims 1-7, wherein the first antigen-binding site comprises a heavy chain variable domain amino acid sequence at least 90% identical to SEQ ID NO:47 and a light chain variable domain amino acid sequence at least 90% identical to SEQ ID NO:48.

13. The protein according to any one of claims 1-7, wherein the first antigen-binding site comprises a heavy chain variable domain amino acid sequence at least 90% identical to SEQ ID NO:49 and a light chain variable domain amino acid sequence at least 90% identical to SEQ ID NO:50.

14. The protein according to any one of claims 1-7, wherein the first antigen-binding site comprises a heavy chain variable domain amino acid sequence at least 90% identical to SEQ ID NO:132 and a light chain variable domain amino acid sequence at least 90% identical to SEQ ID NO:133.

15. The protein according to any one of claims 1-7, wherein the first antigen-binding site comprises a heavy chain variable domain amino acid sequence at least 90% identical to SEQ ID NO:140 and a light chain variable domain amino acid sequence at least 90% identical to SEQ ID NO:141.

16. The protein according to any one of claims 1-7, wherein the first antigen-binding site comprises a heavy chain variable domain amino acid sequence at least 90% identical to SEQ ID NO:148 and a light chain variable domain amino acid sequence at least 90% identical to SEQ ID NO:149.

17. The protein according to any one of claims 1-7, wherein the first antigen-binding site comprises a heavy chain variable domain amino acid sequence at least 90% identical to SEQ ID NO:156 and a light chain variable domain amino acid sequence at least 90% identical to SEQ ID NO:157.

18. The protein according to any one of claims 1-7, wherein the first antigen-binding site comprises a heavy chain variable domain amino acid sequence at least 90% identical to SEQ ID NO:164 and a light chain variable domain amino acid sequence at least 90% identical to SEQ ID NO:165.

19. The protein of claim 1 or 2, wherein the first antigen-binding site is a single-domain antibody.

20. The protein of claim 19, wherein the single-domain antibody is a V_{H}H fragment or a V_{NAR} fragment.

21. The protein according to any one of claim 1-2 or 19-20, wherein the second antigen-binding site comprises a heavy chain variable domain and a light chain variable domain.

22. The protein according to claim 21, wherein the heavy chain variable domain and the light chain variable domain of the second antigen-binding site are present on the same polypeptide.

23-40. (canceled)

41. The protein according to any one of claims 1-22, wherein the second antigen-binding site comprises a heavy chain variable domain comprising an amino acid sequence at least 90% identical to SEQ ID NO:195 and a light chain variable domain comprising an amino acid sequence at least 90% identical to SEQ ID NO:196.

42. The protein according to any one of claims 1-22, wherein the second antigen-binding site comprises a heavy chain variable domain comprising an amino acid sequence at least 90% identical to SEQ ID NO:203 and a light chain variable domain comprising an amino acid sequence at least 90% identical to SEQ ID NO:204.

43. The protein according to any one of claims 1-22, wherein the second antigen-binding site comprises a heavy chain variable domain comprising an amino acid sequence at least 90% identical to SEQ ID NO:211 and a light chain variable domain comprising an amino acid sequence at least 90% identical to SEQ ID NO:212.

44-45. (canceled)

46. A protein according to any one of claim 1-4 or 8-20, wherein the second antigen-binding site is a single-domain antibody.

47. The protein of claim 46, wherein the second antigen-binding site is a V_{H}H fragment or a V_{NAR} fragment.

48. A protein according to any one of the preceding claims, wherein the protein comprises a portion of an antibody Fc domain sufficient to bind CD16, wherein the antibody Fc domain comprises hinge and CH2 domains.

49. A protein according to claim 48, wherein the antibody Fc domain comprises hinge and CH2 domains of a human IgG1 antibody.

50. A protein according to claim 48 or 49, wherein the Fc domain comprises an amino acid sequence at least 90% identical to amino acids 234-332 of a human IgG1 antibody.

51. A protein according to any one of claims 48-50, wherein the Fc domain comprises an amino acid sequence at least 90% identical to the Fc domain of human IgG1 and differs at one or more positions selected from the group consisting of Q347, Y349, L351, S354, E356, E357, K360, Q362, S364, T366, L368, K370, N390, K392, T394, D399, S400, D401, F405, Y407, K409, T411, and K439.

52. A formulation comprising a protein according to any one of the preceding claims and a pharmaceutically acceptable carrier.

53. A cell comprising one or more nucleic acids encoding a protein according to any one of claims 1-51.

54. A method of directly and/or indirectly enhancing tumor cell death, the method comprising exposing the tumor cell and a natural killer cell to an effective amount of the protein according to any one of claims 1-51.

55. A method of treating cancer in a patient in need thereof, wherein the method comprises administering to the patient a therapeutically effective amount of the protein according to any one of claims 1-51 or the formulation according to claim 52.

56-59. (canceled)

60. The method of claim 55, wherein the cancer to be treated is selected from the group consisting of gastrointestinal cancer, colorectal cancer, pancreatic cancer, non-small cell lung cancer and breast cancer.

61. (canceled)


SEQUENCE LISTING (236 sequences)

SEQ ID NO: 1 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 3 - protein, 117 aa, Artificial Sequence; identical to SEQ ID NO: 1
  Description of Artificial Sequence Synthetic polypeptide

SEQ ID NO: 4 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 5 - protein, 117 aa, Artificial Sequence; identical to SEQ ID NO: 1
  Description of Artificial Sequence Synthetic polypeptide

SEQ ID NO: 6 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 7 - protein, 117 aa, Artificial Sequence; identical to SEQ ID NO: 1
  Description of Artificial Sequence Synthetic polypeptide

SEQ ID NO: 8 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 9 - protein, 117 aa, Artificial Sequence; identical to SEQ ID NO: 1
  Description of Artificial Sequence Synthetic polypeptide

SEQ ID NO: 10 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 11 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 12 - protein, 107 aa, Artificial Sequence
  Description of Artificial Sequence Synthetic polypeptide
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 13 - protein, 117 aa, Artificial Sequence; identical to SEQ ID NO: 1
  Description of Artificial Sequence Synthetic polypeptide

SEQ ID NO: 14 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 15 - protein, 117 aa, Artificial Sequence; identical to SEQ ID NO: 1
  Description of Artificial Sequence Synthetic polypeptide

SEQ ID NO: 16 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 17 - protein, 117 aa, Artificial Sequence; identical to SEQ ID NO: 1
  Description of Artificial Sequence Synthetic polypeptide

SEQ ID NO: 18 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 19 - protein, 117 aa, Artificial Sequence; identical to SEQ ID NO: 1
  Description of Artificial Sequence Synthetic polypeptide

SEQ ID NO: 20 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 21 - protein, 117 aa, Artificial Sequence; identical to SEQ ID NO: 1
  Description of Artificial Sequence Synthetic polypeptide

SEQ ID NO: 22 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 23 - protein, 117 aa, Artificial Sequence; identical to SEQ ID NO: 1
  Description of Artificial Sequence Synthetic polypeptide

SEQ ID NO: 24 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 25 - protein, 117 aa, Artificial Sequence; identical to SEQ ID NO: 1
  Description of Artificial Sequence Synthetic polypeptide

SEQ ID NO: 26 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 27 - protein, 117 aa, Artificial Sequence; identical to SEQ ID NO: 1
  Description of Artificial Sequence Synthetic polypeptide

SEQ ID NO: 28 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 29 - protein, 117 aa, Artificial Sequence; identical to SEQ ID NO: 1
  Description of Artificial Sequence Synthetic polypeptide

SEQ ID NO: 30 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 31 - protein, 117 aa, Artificial Sequence; identical to SEQ ID NO: 1
  Description of Artificial Sequence Synthetic polypeptide

SEQ ID NO: 32 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 33 - protein, 117 aa, Artificial Sequence; identical to SEQ ID NO: 1
  Description of Artificial Sequence Synthetic polypeptide

SEQ ID NO: 34 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 35 - protein, 117 aa, Artificial Sequence; identical to SEQ ID NO: 1
  Description of Artificial Sequence Synthetic polypeptide

SEQ ID NO: 36 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 37 - protein, 117 aa, Artificial Sequence; identical to SEQ ID NO: 1
  Description of Artificial Sequence Synthetic polypeptide

SEQ ID NO: 38 - protein, 107 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 39 - protein, 117 aa, Artificial Sequence; identical to SEQ ID NO: 1
  Description of Artificial Sequence Synthetic polypeptide

SEQ ID NO: 40 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 41 - protein, 125 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 42 - protein, 113 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 43 - protein, 121 aa, Artificial Sequence
  Description of Artificial Sequence Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 44 - protein, 107 aa, Artificial Sequence
  Description of Artificial Sequence Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 45 - protein, 126 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 46 - protein, 113 aa, Artificial Sequence [sequence omitted]

SEQ ID NO: 47 - protein, 121 aa, Homo sapiens
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 48 - protein, 110 aa, Homo sapiens
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 49 - protein, 115 aa, Homo sapiens
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 50 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 51 - protein, 9 aa, Artificial Sequence
  Description of Artificial Sequence Synthetic peptide
GSFSGYYWS

SEQ ID NO: 52 - protein, 16 aa, Artificial Sequence
  Description of Artificial Sequence Synthetic peptide
EIDHSGSTNYNPSLKS

SEQ ID NO: 53 - protein, 11 aa, Artificial Sequence
  Description of Artificial Sequence Synthetic peptide
ARARGPWSFDP

SEQ ID NO: 54 - protein, 9 aa, Artificial Sequence
  Description of Artificial Sequence Synthetic peptide
GTFSSYAIS

SEQ ID NO: 55 - protein, 17 aa, Artificial Sequence
  Description of Artificial Sequence Synthetic peptide
GIIPIFGTANYAQKFQG

SEQ ID NO: 56 - protein, 18 aa, Artificial Sequence
  Description of Artificial Sequence Synthetic peptide
ARGDSSIRHAYYYYGMDV

SEQ ID NO: 57 - protein, 17 aa, Artificial Sequence
  Description of Artificial Sequence Synthetic peptide
KSSQSVLYSSNNKNYLA

SEQ ID NO: 58 - protein, 7 aa, Artificial Sequence
  Description of Artificial Sequence Synthetic peptide
WASTRES

SEQ ID NO: 59 - protein, 9 aa, Artificial Sequence
  Description of Artificial Sequence Synthetic peptide
QQYYSTPIT

SEQ ID NO: 60 - protein, 11 aa, Artificial Sequence
  Description of Artificial Sequence Synthetic peptide
GSISSSSYYWG

SEQ ID NO: 61 - protein, 16 aa, Artificial Sequence
  Description of Artificial Sequence Synthetic peptide
SIYYSGSTYYNPSLKS

SEQ ID NO: 62 - protein, 13 aa, Artificial Sequence
  Description of Artificial Sequence Synthetic peptide
ARGSDRFHPYFDY

SEQ ID NO: 63 - protein, 11 aa, Artificial Sequence
  Description of Artificial Sequence Synthetic peptide
RASQSVSRYLA

SEQ ID NO: 64 - protein, 7 aa, Artificial Sequence
  Description of Artificial Sequence Synthetic peptide
DASNRAT

SEQ ID NO: 65 - protein, 9 aa, Artificial Sequence
  Description of Artificial Sequence Synthetic peptide
QQFDTWPPT

SEQ ID NO: 66 - protein, 9 aa, Artificial Sequence
  Description of Artificial Sequence Synthetic peptide
GTFSSYAIS

SEQ ID NO: 67 - protein, 17 aa, Artificial Sequence
  Description of Artificial Sequence Synthetic peptide
GIIPIFGTANYAQKFQG

SEQ ID NO: 68 - protein, 19 aa, Artificial Sequence
  Description of Artificial Sequence Synthetic peptide
ARRGRKASGSFYYYYGMDV

SEQ ID NO: 69 - protein, 17 aa, Artificial Sequence
  Description of Artificial Sequence Synthetic peptide
ESSQSLLNSGNQKNYLT

SEQ ID NO: 70 - protein, 7 aa, Artificial Sequence
  Description of Artificial Sequence Synthetic peptide
WASTRES

SEQ ID NO: 71 - protein, 9 aa, Artificial Sequence
  Description of Artificial Sequence Synthetic peptide
QNDYSYPYT

SEQ ID NO: 72 - protein, 119 aa, Artificial Sequence
  Description of Artificial Sequence Synthetic polypeptide
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSS

SEQ ID NO: 73 - protein, 108 aa, Artificial Sequence
  Description of Artificial Sequence Synthetic polypeptide
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIKR

SEQ ID NO: 74 - protein, 7 aa, Artificial Sequence
  Description of Artificial Sequence Synthetic peptide
GFTFSNY

SEQ ID NO: 75 - protein, 6 aa, Artificial Sequence
  Description of Artificial Sequence Synthetic peptide
NSDGGI

SEQ ID NO: 76 - protein, 10 aa, Artificial Sequence
  Description of Artificial Sequence Synthetic peptide
HRSGYFSMDY

SEQ ID NO: 77 - protein, 8 aa, Artificial Sequence
  Description of Artificial Sequence Synthetic peptide
QNVVSAVA

SEQ ID NO: 78 - protein, 7 aa, Artificial Sequence
  Description of Artificial Sequence Synthetic peptide
SASNRYT

SEQ ID NO: 79 - protein, 9 aa, Artificial Sequence
  Description of Artificial Sequence Synthetic peptide
QQYSNYPWT

SEQ ID NO: 80 - protein, 121 aa, Artificial Sequence
  Description of Artificial Sequence Synthetic polypeptide
QVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSS

SEQ ID NO: 81 - protein, 107 aa, Artificial Sequence
  Description of Artificial Sequence Synthetic polypeptide
DIQMTQSPSSLSASVGDRVTITCRASQDINNYLSWYQQKPGKAPKLLIYGASNLQSGVPSRFSGSGSGTDFTLTISSLQPEDFAVYYCQQYYGRPTTFGQGTKVEIK

SEQ ID NO: 82 - protein, 10 aa, Artificial Sequence
  Description of Artificial Sequence Synthetic peptide
GFTFSSYGMS

SEQ ID NO: 83 - protein, 17 aa, Artificial Sequence
  Description of Artificial Sequence Synthetic peptide
GISSLGSTTYYADSVKG

SEQ ID NO: 84 - protein, 12 aa, Artificial Sequence
  Description of Artificial Sequence Synthetic peptide
TGSPGTFMHGDH

SEQ ID NO: 85 - protein, 11 aa, Artificial Sequence
  Description of Artificial Sequence Synthetic peptide
RASQDINNYLS

SEQ ID NO: 86 - protein, 8 aa, Artificial Sequence
  Description of Artificial Sequence Synthetic peptide
YGASNLQS

SEQ ID NO: 87 - protein, 8 aa, Artificial Sequence
  Description of Artificial Sequence Synthetic peptide
QQYYGRPT

SEQ ID NO: 88 - protein, 119 aa, Artificial Sequence
  Description of Artificial Sequence Synthetic polypeptide
EVQLVESGGGLVQPGGSLRLSCAASGFTFSNYYMSWVRQAPGQRLELVSAINSDGGITYYLDSVKGRFTISRDNAKNTLYLQMGSLRAEDMAVYYCARHRSGYFSMDYWGQGTLVTVSS

SEQ ID NO: 89 - protein, 107 aa, Artificial Sequence
  Description of Artificial Sequence Synthetic polypeptide
DIVMTQSPSSLSASVGDRVTITCRASQNVVSAVAWYLQKPGKSPKLLIYSASNRYTGVPDRFTGSGSGTDFTLTISSLQPEDFATYFCQQYSNYPWTFGGGTKLEIK

SEQ ID NO: 90 - protein, 7 aa, Artificial Sequence
  Description of Artificial Sequence Synthetic peptide
GFTFSNY

SEQ ID NO: 91 - protein, 6 aa, Artificial Sequence
  Description of Artificial Sequence Synthetic peptide
NSDGGI

SEQ ID NO: 92 - protein, 10 aa, Artificial Sequence
  Description of Artificial Sequence Synthetic peptide
HRSGYFSMDY

SEQ ID NO: 93 - protein, 8 aa, Artificial Sequence
  Description of Artificial Sequence Synthetic peptide
QNVVSAVA

SEQ ID NO: 94 - protein, 7 aa, Artificial Sequence
  Description of Artificial Sequence Synthetic peptide
SASNRYT

SEQ ID NO: 95 - protein, 9 aa, Artificial Sequence
  Description of Artificial Sequence Synthetic peptide
QQYSNYPWT

SEQ ID NO: 96 - protein, 459 aa, Unknown
  Description of Unknown CAIX polypeptide
MAPLCPSPWLPLLIPAPAPGLTVQLLLSLLLLVPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 97 - protein, 123 aa, Artificial Sequence
  Description of Artificial Sequence Synthetic polypeptide
QVQLKESGPGLVQPSQTLTLTCTVSGFSLSNYDMHWVRQSPGKGLDWMGVIWGNGKTQYNSGLTSRLSISRDTSKSQVFLKMNSLQTEDTAIYFCTRSGYYYDGSYYSLFDYWGQGVMVTVSS

SEQ ID NO: 98 - protein, 111 aa, Artificial Sequence
  Description of Artificial Sequence Synthetic polypeptide
DVVLTQTPPTLSATIGQSVSISCRSSQSLLHSNGNTYLNWLLQRPGQPPQLLIYLVSRLESGVPNRFSGSGSGTDFTLKISGVEAEDLGVYYCVQSTHAPAFGGGTKLELK

SEQ ID NO: 99 - protein, 5 aa, Artificial Sequence
  Description of Artificial Sequence Synthetic peptide
NYDMH

SEQ ID NO: 100 - protein, 16 aa, Artificial Sequence
  Description of Artificial Sequence Synthetic peptide
VIWGNGKTQYNSGLTS

SEQ ID NO: 101 - protein, 15 aa, Artificial Sequence
  Description of Artificial Sequence Synthetic peptide
SGYYYDGSYYSLFDY

SEQ ID NO: 102 - protein, 16 aa, Artificial Sequence
  Description of Artificial Sequence Synthetic peptide
RSSQSLLHSNGNTYLN

SEQ ID NO: 103 - protein, 7 aa, Artificial Sequence
  Description of Artificial Sequence Synthetic peptide
LVSRLES

SEQ ID NO: 104 - protein, 8 aa, Artificial Sequence
  Description of Artificial Sequence Synthetic peptide
VQSTHAPA

SEQ ID NO: 105 - protein, 986 aa, Unknown
  Description of Unknown ANO1 polypeptide
MRVNEKYSTLPAEDRSVHIINICAIEDIGYLPSEGTLLNSLSVDPDAECKYGLYFRDGRRKVDYILVYHHKRPSGNRTLVRRVQHSDTPSGARSVKQDHPLPGKGASLDAGSGEPPMDYHEDDKRFRREEYEGNLLEAGLELERDEDTKIHGVGFVKIHAPWNVLCREAEFLKLKMPTKKMYHINETRGLLKKINSVLQKITDPIQPKVAEHRPQTMKRLSYPFSREKQHLFDLSDKDSFFDSKTRSTIVYEILKRTTCTKAKYSMGITSLLANGVYAAAYPLHDGDYNGENVEFNDRKLLYEEWARYGVFYKYQPIDLVRKYFGEKIGLYFAWLGVYTQMLIPASIVGIIVFLYGCATMDENIPSMEMCDQRHNITMCPLCDKTCSYWKMSSACATARASHLFDNPATVFFSVFMALWAATFMEHWKRKQMRLNYRWDLTGFEEEEEAVKDHPRAEYEARVLEKSLKKESRNKEKRRHIPEESTNKWKQRVKTAMAGVKLTDKVKLTWRDRFPAYLTNLVSIIFMIAVTFAIVLGVIIYRISMAAALAMNSSPSVRSNIRVTVTATAVIINLVVIILLDEVYGCIARWLTKIEVPKTEKSFEERLIFKAFLLKFVNSYTPIFYVAFFKGRFVGRPGDYVYIFRSFRMEECAPGGCLMELCIQLSIIMLGKQLIQNNLFEIGIPKMKKLIRYLKLKQQSPPDHEECVKRKQRYEVDYNLEPFAGLTPEYMEMIIQFGFVTLFVASFPLAPLFALLNNIIEIRLDAKKFVTELRRPVAVRAKDIGIWYNILRGIGKLAVIINAFVISFTSDFIPRLVYLYMYSKNGTMHGFVNHTLSSFNVSDFQNGTAPNDPLDLGYEVQICRYKDYREPPWSENKYDISKDFWAVLAARLAFVIVFQNLVMFMSDFVDWVIPDIPKDISQQIHKEKVLMVELFMREEQDKQQLLETWMEKERQKDEPPCNHHNTKACPDSLGSPAPSHAYHGGVL

SEQ ID NO: 106 - protein, 120 aa, Artificial Sequence
  Description of Artificial Sequence Synthetic polypeptide
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSA

SEQ ID NO: 107 - protein, 107 aa, Artificial Sequence
  Description of Artificial Sequence Synthetic polypeptide
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKR

SEQ ID NO: 108 - protein, 7 aa, Artificial Sequence
  Description of Artificial Sequence Synthetic peptide
GYSFTGY

SEQ ID NO: 109 - protein, 6 aa, Artificial Sequence
  Description of Artificial Sequence Synthetic peptide
TPYNGA

SEQ ID NO: 110 - protein, 10 aa, Artificial Sequence
  Description of Artificial Sequence Synthetic peptide
GGYDGRGFDY

SEQ ID NO: 111 - protein, 7 aa, Artificial Sequence
  Description of Artificial Sequence Synthetic peptide
SSVSYMH

SEQ ID NO: 112 - protein, 7 aa, Artificial Sequence
  Description of Artificial Sequence Synthetic peptide
DTSKLAS

SEQ ID NO: 113 - protein, 9 aa, Artificial Sequence
  Description of Artificial Sequence Synthetic peptide
QQWSKHPLT

SEQ ID NO: 114 - protein, 131 aa, Artificial Sequence
  Description of Artificial Sequence Synthetic polypeptide
EVHLVESGGGLVQPGGSLRLSCAASGFTFSRYWMSWVRQAQGKGLEWVASIKQAGSEKTYVDSVKGRFTISRDNAKNSLSLQMNSLRAEDTAVYYCAREGAYYYDSASYYPYYYYYSMDVWGQGTTVTVSS

SEQ ID NO: 115 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSQYTFGQGTKLEIK

SEQ ID NO: 116 - protein, 7 aa, Artificial Sequence
  Description of Artificial Sequence Synthetic peptide
GFTFSRY

SEQ ID NO: 117 - protein, 6 aa, Artificial Sequence
  Description of Artificial Sequence Synthetic peptide
KQAGSE

SEQ ID NO: 118 - protein, 22 aa, Artificial Sequence
  Description of Artificial Sequence Synthetic peptide
EGAYYYDSASYYPYYYYYSMDV

SEQ ID NO: 119 - protein, 9 aa, Artificial Sequence
  Description of Artificial Sequence Synthetic peptide
QSVSSSYLA

SEQ ID NO: 120 - protein, 7 aa, Artificial Sequence
  Description of Artificial Sequence Synthetic peptide
GASSRAT

SEQ ID NO: 121 - protein, 9 aa, Artificial Sequence
  Description of Artificial Sequence Synthetic peptide
QQYGSSQYT

SEQ ID NO: 122 - protein, 121 aa, Artificial Sequence
  Description of Artificial Sequence Synthetic polypeptide
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSA

SEQ ID NO: 123 - protein, 112 aa, Artificial Sequence
  Description of Artificial Sequence Synthetic polypeptide
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLG

SEQ ID NO: 124 - protein, 7 aa, Artificial Sequence
  Description of Artificial Sequence Synthetic peptide
GYSFTSY

SEQ ID NO: 125 - protein, 6 aa, Artificial Sequence
  Description of Artificial Sequence Synthetic peptide
DPGDSR

SEQ ID NO: 126 - protein, 11 aa, Artificial Sequence
  Description of Artificial Sequence Synthetic peptide
GQLYGGTYMDG

SEQ ID NO: 127 - protein, 11 aa, Artificial Sequence
  Description of Artificial Sequence Synthetic peptide
SSDIGGYNSVS

SEQ ID NO: 128 - protein, 7 aa, Artificial Sequence
  Description of Artificial Sequence Synthetic peptide
GVNNRPS

SEQ ID NO: 129 - protein, 11 aa, Artificial Sequence
  Description of Artificial Sequence Synthetic peptide
SSYDIESATPV

SEQ ID NO: 130 - protein, 630 aa, Unknown
  Description of Unknown mesothelin polypeptide
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 131 - protein, 246 aa, Unknown
  Description of Unknown ULBP-6 polypeptide
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 132 - protein, 121 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 133 - protein, 107 aa, Artificial Sequence
  Description of Artificial Sequence Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 134 - protein, 9 aa, Artificial Sequence
  Description of Artificial Sequence Synthetic peptide
FTFSSYAMS

SEQ ID NO: 135 - protein, 17 aa, Artificial Sequence
  Description of Artificial Sequence Synthetic peptide
AISGSGGSTYYADSVKG

SEQ ID NO: 136 - protein, 14 aa, Artificial Sequence
  Description of Artificial Sequence Synthetic peptide
AKDGGYYDSGAGDY

SEQ ID NO: 137 - protein, 11 aa, Artificial Sequence
  Description of Artificial Sequence Synthetic peptide
RASQGIDSWLA

SEQ ID NO: 138 - protein, 7 aa, Artificial Sequence
  Description of Artificial Sequence Synthetic peptide
AASSLQS

SEQ ID NO: 139 - protein, 9 aa, Artificial Sequence
  Description of Artificial Sequence Synthetic peptide
QQGVSYPRT

SEQ ID NO: 140 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 141 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 142 - protein, 9 aa, Artificial Sequence
  Description of Artificial Sequence Synthetic peptide
FTFSSYSMN

SEQ ID NO: 143 - protein, 17 aa, Artificial Sequence
  Description of Artificial Sequence Synthetic peptide
SISSSSSYIYYADSVKG

SEQ ID NO: 144 - protein, 15 aa, Artificial Sequence
  Description of Artificial Sequence Synthetic peptide
ARGAPMGAAAGWFDP

SEQ ID NO: 145 - protein, 11 aa, Artificial Sequence
  Description of Artificial Sequence Synthetic peptide
RASQGISSWLA

SEQ ID NO: 146 - protein, 7 aa, Artificial Sequence
  Description of Artificial Sequence Synthetic peptide
AASSLQS

SEQ ID NO: 147 - protein, 9 aa, Artificial Sequence
  Description of Artificial Sequence Synthetic peptide
QQGVSFPRT

SEQ ID NO: 148 - protein, 124 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 149 - protein, 107 aa, Artificial Sequence
  Description of Artificial Sequence Synthetic polypeptide
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 150 - protein, 9 aa, Artificial Sequence
  Description of Artificial Sequence Synthetic peptide
YTFTGYYMH

SEQ ID NO: 151 - protein, 17 aa, Artificial Sequence
  Description of Artificial Sequence Synthetic peptide
WINPNSGGTNYAQKFQG

SEQ ID NO: 152 - protein, 17 aa, Artificial Sequence
  Description of Artificial Sequence Synthetic peptide
ARDTGEYYDTDDHGMDV

SEQ ID NO: 153 - protein, 11 aa, Artificial Sequence
  Description of Artificial Sequence Synthetic peptide
RASQSVSSNLA

SEQ ID NO: 154 - protein, 7 aa, Artificial Sequence
  Description of Artificial Sequence Synthetic peptide
GASTRAT

SEQ ID NO: 155 - protein, 9 aa, Artificial Sequence
  Description of Artificial Sequence Synthetic peptide
QQDDYWPPT

SEQ ID NO: 156 - protein, 126 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 157 - protein, 107 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 158 - protein, 9 aa, Artificial Sequence
  Description of Artificial Sequence Synthetic peptide
YTFTSYYMH

SEQ ID NO: 159 - protein, 17 aa, Artificial Sequence
  Description of Artificial Sequence Synthetic peptide
IINPSGGSTSYAQKFQG

SEQ ID NO: 160 - protein, 19 aa, Artificial Sequence
  Description of Artificial Sequence Synthetic peptide
ARGAPNYGDTTHDYYYMDV

SEQ ID NO: 161 - protein, 11 aa, Artificial Sequence
  Description of Artificial Sequence Synthetic peptide
RASQSVSSNLA

SEQ ID NO: 162 - protein, 7 aa, Artificial Sequence
  Description of Artificial Sequence Synthetic peptide
GASTRAT

SEQ ID NO: 163 - protein, 9 aa, Artificial Sequence
  Description of Artificial Sequence Synthetic peptide
QQYDDWPFT

SEQ ID NO: 164 - protein, 117 aa, Artificial Sequence; identical to SEQ ID NO: 1
  Description of Artificial Sequence Synthetic polypeptide

SEQ ID NO: 165 - protein, 106 aa, Artificial Sequence; identical to SEQ ID NO: 40
  Description of Artificial Sequence Synthetic polypeptide

SEQ ID NO: 166 - protein, 9 aa, Artificial Sequence
  Description of Artificial Sequence Synthetic peptide
GSFSGYYWS

SEQ ID NO: 167 - protein, 16 aa, Artificial Sequence
  Description of Artificial Sequence Synthetic peptide
EIDHSGSTNYNPSLKS

SEQ ID NO: 168 - protein, 11 aa, Artificial Sequence
  Description of Artificial Sequence Synthetic peptide
ARARGPWSFDP

SEQ ID NO: 169 - protein, 11 aa, Artificial Sequence
  Description of Artificial Sequence Synthetic peptide
RASQSISSWLA

SEQ ID NO: 170 - protein, 7 aa, Artificial Sequence
  Description of Artificial Sequence Synthetic peptide
KASSLES

SEQ ID NO: 171 - protein, 8 aa, Artificial Sequence
  Description of Artificial Sequence Synthetic peptide
QQYDTFIT

SEQ ID NO: 172 - protein, 121 aa, Artificial Sequence
  Description of Artificial Sequence Synthetic polypeptide
SVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 173 - protein, 108 aa, Artificial Sequence
  Description of Artificial Sequence Synthetic polypeptide
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 174 - protein, 7 aa, Artificial Sequence
  Description of Artificial Sequence Synthetic peptide
GYTFTNY

SEQ ID NO: 175 - protein, 6 aa, Artificial Sequence
  Description of Artificial Sequence Synthetic peptide
NTYTGE

SEQ ID NO: 176 - protein, 12 aa, Artificial Sequence
  Description of Artificial Sequence Synthetic peptide
GGFGSSYWYFDV

SEQ ID NO: 177 - protein, 8 aa, Artificial Sequence
  Description of Artificial Sequence Synthetic peptide
QDVSIAVA

SEQ ID NO: 178 - protein, 7 aa, Artificial Sequence
  Description of Artificial Sequence Synthetic peptide
SASYRYT

SEQ ID NO: 179 - protein, 9 aa, Artificial Sequence
  Description of Artificial Sequence Synthetic peptide
QQHYITPLT

SEQ ID NO: 180 - protein, 121 aa, Artificial Sequence
  Description of Artificial Sequence Synthetic polypeptide
QIQLVQSGHEVKKPGASVKVSCKASGYTFTNYGMNWVRQAPGQGLEWMGWINTKTGEPTYAEEFKGRFVFSLETSASTAYLQISSLKAEDTAMYFCGRGGYGSSYWYFDVWGQGTTVTVSS

SEQ ID NO: 181 - protein, 107 aa, Artificial Sequence
  Description of Artificial Sequence Synthetic polypeptide
DIQMTQSPSSLSASVGDRVTITCKASQDVSIAVAWYQQKPGKAPKVLIYSASYRYTGVPDRFSGSGSGTDFTFTISSLQPEDIAVYYCQQHYITPLTFGGGTKVEIK

SEQ ID NO: 182 - protein, 7 aa, Artificial Sequence
  Description of Artificial Sequence Synthetic peptide
GYTFTNY

SEQ ID NO: 183 - protein, 6 aa, Artificial Sequence
  Description of Artificial Sequence Synthetic peptide
NTKTGE

SEQ ID NO: 184 - protein, 12 aa, Artificial Sequence
  Description of Artificial Sequence Synthetic peptide
GGYGSSYWYFDV

SEQ ID NO: 185 - protein, 8 aa, Artificial Sequence
  Description of Artificial Sequence Synthetic peptide
QDVSIAVA

SEQ ID NO: 186 - protein, 7 aa, Artificial Sequence
  Description of Artificial Sequence Synthetic peptide
SASYRYT

SEQ ID NO: 187 - protein, 9 aa, Artificial Sequence
  Description of Artificial Sequence Synthetic peptide
QQHYITPLT

SEQ ID NO: 188 - protein, 121 aa, Artificial Sequence
  Description of Artificial Sequence Synthetic polypeptide
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 189 - protein, 109 aa, Artificial Sequence
  Description of Artificial Sequence Synthetic polypeptide
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRT

SEQ ID NO: 190 - protein, 121 aa, Artificial Sequence
  Description of Artificial Sequence Synthetic polypeptide
QVQLVQSGAEVKKPGASVKVSCKASGYTFTTAGMQWVRQAPGQGLEWMGWINTHSGVPKYAEDFKGRVTISLDTSTSTAYLQLSSLKSEDTAVYYCARSGFGSSYWYFDVWGQGTLVTVSS

SEQ ID NO: 191 - protein, 109 aa, Artificial Sequence
  Description of Artificial Sequence Synthetic polypeptide
DIVMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRT

SEQ ID NO: 192 - protein, 121 aa, Artificial Sequence
  Description of Artificial Sequence Synthetic polypeptide
QIQLVQSGAEVKKPGESVKVSCKASGYTFTTAGMQWVQQMPGKGLEWMGWINTHSGVPKYAEDFKGRVTFSLDTSTSTAYLQLSSLKSEDTAVYYCARSGFGSSYWYFDVWGQGTLVTVSS

SEQ ID NO: 193 - protein, 108 aa, Artificial Sequence
  Description of Artificial Sequence Synthetic polypeptide
DIVMTQSPSSLSASVGDRVTITCKASQDVSTAVAWYQQKPGKQPKLLIYSASYRYTGVPSRFSGSGSGTDFTLTISSLQPEDFAVYYCQQHYITPLTFGQGTKLEIKR

SEQ ID NO: 194 - protein, 323 aa, Unknown
  Description of Unknown TROP2 polypeptide
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 195 - protein, 119 aa, Artificial Sequence
  Description of Artificial Sequence Synthetic polypeptide
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 196 - protein, 106 aa, Artificial Sequence
  Description of Artificial Sequence Synthetic polypeptide
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 197 - protein, 7 aa, Artificial Sequence
  Description of Artificial Sequence Synthetic peptide
GFDFTTY

SEQ ID NO: 198 - protein, 6 aa, Artificial Sequence
  Description of Artificial Sequence Synthetic peptide
HPDSST

SEQ ID NO: 199 - protein, 10 aa, Artificial Sequence
  Description of Artificial Sequence Synthetic peptide
LYFGFPWFAY

SEQ ID NO: 200 - protein, 8 aa, Artificial Sequence
  Description of Artificial Sequence Synthetic peptide
QDVGTSVA

SEQ ID NO: 201 - protein, 7 aa, Artificial Sequence
  Description of Artificial Sequence Synthetic peptide
WTSTRHT

SEQ ID NO: 202 - protein, 8 aa, Artificial Sequence
  Description of Artificial Sequence Synthetic peptide
QQYSLYRS

SEQ ID NO: 203 - protein, 122 aa, Artificial Sequence
  Description of Artificial Sequence Synthetic polypeptide
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSA

SEQ ID NO: 204 - protein, 109 aa, Artificial Sequence
  Description of Artificial Sequence Synthetic polypeptide
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKR

SEQ ID NO: 205 - protein, 7 aa, Artificial Sequence
  Description of Artificial Sequence Synthetic peptide
GYTFTEF

SEQ ID NO: 206 - protein, 6 aa, Artificial Sequence
  Description of Artificial Sequence Synthetic peptide
NTKTGE

SEQ ID NO: 207 - protein, 12 aa, Artificial Sequence
  Description of Artificial Sequence Synthetic peptide
WDFAYYVEAMDY

SEQ ID NO: 208 - protein, 8 aa, Artificial Sequence
  Description of Artificial Sequence Synthetic peptide
AAVGTYVA

SEQ ID NO: 209 - protein, 7 aa, Artificial Sequence
  Description of Artificial Sequence Synthetic peptide
SASYRKR

SEQ ID NO: 210 - protein, 10 aa, Artificial Sequence
  Description of Artificial Sequence Synthetic peptide
HQYYTYPLFT

SEQ ID NO: 211 - protein, 121 aa, Artificial Sequence
  Description of Artificial Sequence Synthetic polypeptide
QVQLQQSGAELVRSGASIKLSCTASGFNIKHYYMHWVKQRPEQGLEWIGWINPENVDTEYAPKFQGKATMTADTSSNTAYLQLSSLTSEDTAVYYCNHYRYAGGGALDYWGQGTTVTVSSA

SEQ ID NO: 212 - protein, 108 aa, Artificial Sequence
  Description of Artificial Sequence Synthetic polypeptide
DIQLTQSPAIMSASPGEKVTMTCSASSSVSYIHWYQQKSGTSPKRWVYDTSKLASGVPARFSGSGSGTSYSLTISTMEAEDAATYYCQQWNNNPYSFGGGTKVVIKTV

SEQ ID NO: 213 - protein, 7 aa, Artificial Sequence
  Description of Artificial Sequence Synthetic peptide
GFNIKHY

SEQ ID NO: 214 - protein, 6 aa, Artificial Sequence
  Description of Artificial Sequence Synthetic peptide
NPENVD

SEQ ID NO: 215 - protein, 11 aa, Artificial Sequence
  Description of Artificial Sequence Synthetic peptide
YRYAGGGALDY

SEQ ID NO: 216 - protein, 7 aa, Artificial Sequence
  Description of Artificial Sequence Synthetic peptide
SSVSYIH

SEQ ID NO: 217 - protein, 7 aa, Artificial Sequence
  Description of Artificial Sequence Synthetic peptide
DTSKLAS

SEQ ID NO: 218 - protein, 9 aa, Artificial Sequence
  Description of Artificial Sequence Synthetic peptide
QQWNNNPYS

SEQ ID NO: 219 - protein, 702 aa, Unknown
  Description of Unknown CEA polypeptide
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 220 - protein, 118 aa, Artificial Sequence
  Description of Artificial Sequence Synthetic polypeptide
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSS

SEQ ID NO: 221 - protein, 113 aa, Artificial Sequence
  Description of Artificial Sequence Synthetic polypeptide
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 222 - protein, 8 aa, Artificial Sequence
  Description of Artificial Sequence Synthetic peptide
GYTFTNYG

SEQ ID NO: 223 - protein, 8 aa, Artificial Sequence
  Description of Artificial Sequence Synthetic peptide
INTNTGEP

SEQ ID NO: 224 - protein, 11 aa, Artificial Sequence
  Description of Artificial Sequence Synthetic peptide
ARLGFGNAMDY

SEQ ID NO: 225 - protein, 12 aa, Artificial Sequence
  Description of Artificial Sequence Synthetic peptide
QSLLNSGNQKNY

SEQ ID NO: 226 - protein, 3 aa, Artificial Sequence
  Description of Artificial Sequence Synthetic peptide
WAS

SEQ ID NO: 227 - protein, 9 aa, Artificial Sequence
  Description of Artificial Sequence Synthetic peptide
QNDYSYPLT

SEQ ID NO: 228 - protein, 118 aa, Artificial Sequence
  Description of Artificial Sequence Synthetic polypeptide
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 229 - protein, 113 aa, Artificial Sequence
  Description of Artificial Sequence Synthetic polypeptide
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 230 - protein, 8 aa, Artificial Sequence
  Description of Artificial Sequence Synthetic peptide
GYTFTSYW

SEQ ID NO: 231 - protein, 8 aa, Artificial Sequence
  Description of Artificial Sequence Synthetic peptide
IYPSDSYT

SEQ ID NO: 232 - protein, 11 aa, Artificial Sequence
  Description of Artificial Sequence Synthetic peptide
TRSWRGNSFDY

SEQ ID NO: 233 - protein, 12 aa, Artificial Sequence
  Description of Artificial Sequence Synthetic peptide
QSLLNSGNQKNY

SEQ ID NO: 234 - protein, 3 aa, Artificial Sequence
  Description of Artificial Sequence Synthetic peptide
WAS

SEQ ID NO: 235 - protein, 9 aa, Artificial Sequence
  Description of Artificial Sequence Synthetic peptide
QNDYSYPFT

SEQ ID NO: 236 - protein, 261 aa, Unknown
  Description of Unknown Claudin-18.2 polypeptide
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV